(19) United States
(12) Patent Application Publication
(10) Pub. No.: US 2021/0361563 A1
(43) Pub. Date: Nov. 25, 2021

(54) TRANSDERMAL SYSTEM FOR SYNERGISTIC IMMUNE-CHEMOTHERAPY USING MICRONEEDLES AND METHOD OF TREATMENT THEREOF

(71) Applicant: THE UNIVERSITY OF HONG KONG, Hong Kong (CN)

(72) Inventors: Yu-xiong SU, Hong Kong (CN); Xinmiao LAN, Hong Kong (CN); Xi XIE, Guangzhou (CN)

(21) Appl. No.: 17/155,251

(22) Filed: Jan. 22, 2021

Related U.S. Application Data
(60) Provisional application No. 63/028,850, filed on May 22, 2020.

(51) Int. Cl. A61K 9/00; A61K 33/243; A61K 39/395; A61K 9/51; C07K 16/28; A61P 35/00

(52) U.S. Cl. CPC A61K 9/0021; A61K 33/243; A61K 39/39541; A61K 2039/505; A61K 9/5138; C07K 16/2818; A61P 35/00; A61K 9/5123

(57) ABSTRACT

Provided herein is a transdermal system for direct local co-delivery of immune checkpoint inhibitor and chemotherapeutic agent by microneedles for synergistic immuno-chemotherapy. Provided herein are lipid-coated nanoparticles and their use to facilitate the drug release and tumor-targeting. Also provided herein is a method of treatment of cancer and other disorder using the system. The present treatment method boost immune response in the tumor microenvironment and enhance the inhibition efficiency for tumor cells and decrease systemic toxicity in a subject unresponsive to systemic therapy.

### RELATED APPLICATIONS

The present application claims priority to U.S. Provisional Application Ser. No. 63/028,850, filed May 22, 2020 and incorporate by reference in its entirety.

### 1. FIELD

Provided herein is a transdermal system for direct local co-delivery of immune checkpoint inhibitor and chemotherapeutic agent by microneedles for synergistic immuno-chemotherapy. Provided herein are lipid-coated nanoparticles and their use to facilitate the drug release and tumor-targeting. Also provided herein is a method of treatment of cancer and other disorder using the system.

The patent or application file contains at least one drawing executed in color. Copies of this patent or patent application publication with color drawings will be provided by the Office upon request and payment of the necessary fee.

### 2. BACKGROUND

Cancer is the second leading cause of deaths after cardiovascular diseases. Although chemotherapy remains the main strategy for cancer therapy, immune checkpoint inhibitors have led a paradigm shift in cancer treatment recently. Programmed cell death protein-1 (PD-1) expressed on T-cells binds to its receptor programmed cell death ligand-1 (PD-L1) on tumor cells, leading to inactivation of T-cell immune responses and contributing to tumor immune evasion.¹ The immune checkpoint inhibitor anti-PD-1 (aPD-1) can block the binding of PD-1 to PD-L1, thereby activating the T-cell immune responses to tumor cells.² The application of immune checkpoint inhibitors has rapidly increased for a wide spectrum of cancers and has demonstrated encouraging treatment outcomes.³ However, the limitations of immune checkpoint inhibitors hinder their further clinical application. The overall objective response rate to aPD-1 was reported to be approximately 33% in advanced melanoma,⁴ and only 20% in head and neck cancer.⁵ A high percentage of patients exhibited primary resistance or developed adaptive resistance during the treatment course.^{6, 7, 8} Furthermore, the immune response activated by aPD-1 is usually not persistent.^{2, 9} Improving the response rate and overcoming the resistance remain major challenges in immunotherapy.

Immune profiling in the tumor microenvironment plays a critical role in chemotherapy and immunotherapy. Clinical studies demonstrated that stromal fibroblasts in the tumor microenvironment confer cis-diammineplatinium(II) (CDDP) chemo-resistance, and CD8+ T cells abolish the cancer-associated fibroblast-mediated chemo-resistance through CD8+ T-cell-derived interferon(IFN)-γ (a major effector cytokine of CD8+ T-cell), which reveals the interplay between chemotherapy and immunotherapy.¹⁰⁻¹² The clonal expansion of T-cells in the tumor microenvironment correlates with a better response to chemotherapy;¹³ therefore, the effectiveness of chemotherapy relies on the induction of a durable anticancer immune response.^{14, 15} Pre-existing CD8+ T-cells in the tumor microenvironment also predict the efficacy of aPD-1 therapy. This represents a novel treatment strategy in which the outcome of chemotherapy and immunotherapy can be improved by altering the tumor microenvironment. It has been reported that combination therapies are potentially synergistic and more effective than monotherapies to combat resistance, because tumors evade the immune response through multiple pathways.^{1, 16}

Immunotherapy using immune checkpoint inhibitor anti-PD-1 (aPD-1) has revolutionized cancer treatment. However, the limitations of immune checkpoint inhibitors hinder their further clinical application. The overall objective response rate to aPD-1 was found to be around 33% in advanced melanoma and only 20% in HNC. A high percentage of patients showed primary resistance or developed adaptive resistance during the treatment. Furthermore, the immune response activated by aPD-1 was usually not durable. Improving the response rate and overcoming the resistance are still major challenges in immunotherapy.

### 3. SUMMARY

In one embodiment, provided herein is a new concept for the direct local co-delivery of immune checkpoint inhibitor and cisplatin (CDDP, a chemotherapeutic agent) by microneedles for synergistic immuno-chemotherapy. The is a novel treatment strategy to increase the treatment outcomes in life-threatening cancers. In one embodiment, the treatment is directed to subject that are resistant to one or more cancer therapies. In one embodiment, the microneedle delivers aPD-1 and CDDP. In one embodiment, provided herein is a direct local co-delivery of immune checkpoint inhibitor and chemotherapeutic agent by microneedles for synergistic immuno-chemotherapy.

In one embodiment, the robust T cell response activated by MNs can enhance the efficacy of both aPD-1 and CDDP. Synergistic anticancer mechanisms are activated via robust T cells response induced by microneedle, blockage of PD-1 in T cells by aPD-1, and direct cytotoxicity of CDDP in tumour cells. This method synergistically enhanced anticancer effects. MNs piercing the immune-cell-rich epidermis provoked robust immune responses by activating T-cells. Synergistic immuno-chemotherapeutic effects were sustained through robust T-cells response activated by MNs, blockage of PD-1 in T cells by aPD-1, and direct cytotoxicity of CDDP in tumor cells. The MN-mediated host immune response augmented the aPD-1-activated T-cell immunity, which enhanced the direct killing of cancer cells by CDDP.

In one embodiment, the method provided herein is an antibody-dependent cell-mediated cytotoxicity or “ADCC” which refers to a form of cytotoxicity in which secreted immunoglobulins bound onto Fc receptors (FcRs) present on certain cytotoxic cells (e.g., Natural Killer (NK) cells, neutrophils, and macrophages) enable these cytotoxic effector cells to bind specifically to an antigen-bearing target cell and subsequently kill the target cell with cytotoxins. Specific high-affinity IgG antibodies directed to the surface of target cells “arm” the cytotoxic cells and are absolutely required for such killing. Lysis of the target cell is extracellular, requires direct cell-to-cell contact, and does not involve complement. It is contemplated that, in addition to antibodies, other proteins comprising Fc regions, specifically Fc fusion proteins, having the capacity to bind specifically to an antigen-bearing target cell will be able to effect cell-mediated cytotoxicity. For simplicity, the cell-mediated cytotoxicity resulting from the activity of an Fc fusion protein is also referred to herein as ADCC activity.

Provided herein is a transdermal drug delivery system comprising microneedles, said microneedles comprises nanoparticles that comprise anti-PD-1-cisplatin.

In one embodiment, the nanoparticles are pH-responsive.

In one embodiment, the nanoparticles comprise a lipid and polyvinylpyrrolidone (PVP).

In one embodiment, the microneedles are water soluble.

Provided herein is a method of treatment for cancer in a subject in need thereof comprising administering a transdermal drug delivery system to the subject, said system comprising microneedles and wherein the microneedles comprise a therapeutically effective dose of anti-PD-1-cisplatin-nanoparticles.

In one embodiment, the treatment has a synergistic effect as compared to administration of either anti-PD-1 or cisplatin (CDDP).

In one embodiment, the cancer is melanoma, head and neck cancer or squamous cell carcinoma.

In one embodiment, the method further comprises administration of an additional therapy.

In one embodiment, prior to treatment, the subject exhibits primary resistance, developed adaptive resistance and/or unresponsive to chemotherapy.

In one embodiment, the treatment boosted an immune response and/or reduced tumor volume by at least 8-fold as compared with systemic injection of anti-PD-1.

In one embodiment, the treatment inhibits cell proliferation and/or enhance T-cell infiltration.

In certain embodiments, provided herein is a microneedle which is formed of biocompatible copolymers containing nanoparticles comprising one or more therapeutics. The microneedle can deliver nanoparticles comprising one or more water-soluble or hydrophobic drug while being carried in the microneedle. In particular, since a fat-soluble drug is delivered while being carried by micelle-type self-assembled nanoparticles which are formed as the structure is dissolved, it is possible to greatly increase the solubility in an aqueous solution. As such, existing drugs with poor absorption can be delivered through the skin of a body. In one embodiment, the nanoparticle comprises 1,2-dioleoyl-sn-glycerol-3-phospate (DOPA) formed a bilayer to enable linking with other lipids, such as 1,2-dioleoyl-3-trimethy-lammonium-propane (DOTAP), 1,2-distearoyl-sn-glycero-3-phosphoethanolamine-N-[amino(polyethylene glycol)-20001] (ammonium salt) (DSPE-PEG-AA) and cholesterol.

In one embodiment, the delivery system is a transdermal drug delivery system. In certain embodiments, the transdermal drug delivery system comprises microneedles, said microneedles comprise nanoparticles that comprise anti-PD-1 and cisplatin.

In certain embodiments, the anti-PD-1 is an antibody or PD-1 antigen binding fragments thereof.

The term “antibody” means an immunoglobulin molecule that recognizes and specifically binds to a target, such as a protein, polypeptide, peptide, carbohydrate, polynucleotide, lipid, or combinations of the foregoing through at least one antigen recognition site within the variable region of the immunoglobulin molecule. As used herein, the term “antibody” encompasses intact polyclonal antibodies, intact monoclonal antibodies, antibody fragments (such as Fab, Fab′, F(ab′)2, and Fv fragments), single chain Fv (scFv) mutants, multispecific antibodies such as bispecific antibodies generated from at least two intact antibodies, chimeric antibodies, humanized antibodies, human antibodies, fusion proteins comprising an antigen determination portion of an antibody, and any other modified immunoglobulin molecule comprising an antigen recognition site so long as the antibodies exhibit the desired biological activity.

An antibody can be of any the five major classes of immunoglobulins: IgA, IgD, IgE, IgG, and IgM, or subclasses (isotypes) thereof (e.g. IgG1, IgG2, IgG3, IgG4, IgA1 and IgA2), based on the identity of their heavy-chain constant domains referred to as alpha, delta, epsilon, gamma, and mu, respectively. The different classes of immunoglobulins have different and well-known subunit structures and three-dimensional configurations. Antibodies can be naked or conjugated to other molecules such as toxins, radioisotopes, etc. to form ADCs.

An anti-PD-1 antibody is an antibody or antigen binding fragment thereof that selectively binds a PD-1 polypeptide. Exemplary anti-PD-1 antibodies include for example pembrolizumab (KEYTRUDA.RTM., lambrolizumab, MK-3475), nivolumab (OPDIVA.RTM., BMS-936558, MDX-1106, ONO-4538), or AMP-224.

The term “antigen binding fragment” refers to a molecule comprising a portion of an intact antibody, and in particular refers to a molecule comprising the antigenic determining variable regions of an intact antibody. It is known in the art that the antigen binding function of an antibody can be performed by fragments of a full-length antibody. Examples of antibody fragments include, but are not limited to Fab, Fab′, F(ab′)2, and Fv fragments, linear antibodies, single chain antibodies, and multispecific antibodies formed from antibody fragments.

In certain embodiments, the antigen-binding fragment is Fv, Fab, F(ab′)2, Fab′, dsFv, scFv, or sc(Fv)2.

In certain embodiments, the antibody or antigen-binding fragment thereof is conjugated to at least one heterologous agent, including for example an anticancer agent.

In certain embodiments, the nanoparticles are pH-responsive. In one embodiment, the nanoparticles are self-assembled.

In certain embodiments, nanoparticles are effective to reduce side effects, enhance active tumor focusing, improve the cellular uptake, and nuclear/cytoplasmic targeting of chemotherapy and immunotherapy.

In certain embodiments, the nanoparticles comprise a lipid and polyvinylpyrrolidone (PVP).

Provided herein is a method of making anti-PD-1-CDDP nanoparticles.

In certain embodiments, the microneedles are water soluble and could be dissolved in the tissue.

In certain embodiments, the microneedles comprise of 9×9 needles with 800 μm in height and a base diameter of approximately 400 μm.

Provided herein is a kit comprising a transdermal system for the treatment of cancer or a proliferative disorder. The kit comprises a transdermal system and instructions for using the system. The kit comprises a transdermal system comprising microneedles, said microneedles further comprising anti-PD-1-CDDP nanoparticles.

Provided herein is a method of inhibiting cell growth comprising administering a transdermal drug delivery system to the subject, said system comprising microneedles and wherein the microneedles comprise an effective dose of anti-PD-1-CDDP-nanoparticles.

Provides herein is a method of inhibiting tumor growth in a subject, the method involving administering to a subject in need thereof an effective dose of anti-PD-1-CDDP-nanoparticles.

Provides herein is a method of increasing an anti-tumor immune response in a subject, the method involving administering to a subject in need thereof an effective dose of anti-PD-1-CDDP-nanoparticles.

Provided herein is a method of treatment for cancer in a subject in need thereof comprising administering a transdermal drug delivery system to the subject, said system comprises microneedles and wherein the microneedles comprise a therapeutically effective dose of anti-PD-1-CDDP-nanoparticles.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. In certain aspects, a subject is successfully “treated” for cancer according to the methods of the present disclosure if the patient shows, e.g., total, partial, or transient remission of a certain type of cancer.

An “effective amount” can be determined empirically and in a routine manner, in relation to the stated purpose.

In certain embodiments, the treatment has a synergistic effect as compared to administration of either anti-PD-1 or cisplatin (CDDP).

The terms “cancer”, “tumor”, “cancerous”, and “malignant” refer to or describe the physiological condition in mammals that is typically characterized by unregulated cell growth. Examples of cancers include but are not limited to, carcinoma including adenocarcinomas, lymphomas, blastomas, melanomas, sarcomas, and leukemias. More particular examples of such cancers include squamous cell cancer, small-cell lung cancer, non-small cell lung cancer, gastrointestinal cancer, Hodgkin's and non-Hodgkin's lymphoma, pancreatic cancer, glioblastoma, glioma, cervical cancer, ovarian cancer, liver cancer such as hepatic carcinoma and hepatoma, bladder cancer, breast cancer (including hormonally mediated breast cancer, colon cancer, colorectal cancer, endometrial carcinoma, myeloma (such as multiple myeloma), salivary gland carcinoma, kidney cancer such as renal cell carcinoma and Wilms' tumors, basal cell carcinoma, melanoma, prostate cancer, vulval cancer, thyroid cancer, testicular cancer, esophageal cancer, various types of head and neck cancer and cancers of mucinous origins, such as, mucinous ovarian cancer, cholangiocarcinoma (liver) and renal papillary carcinoma. In certain embodiments, the cancer is selected from colon cancer, breast cancer, lymphoma, and non-small cell carcinoma.

In certain embodiments, the cancer is selected from colorectal cancer, pancreatic cancer, bladder cancer, leukemia, lymphoma, glioma, glioblastoma, melanoma, ovarian cancer, thyroid cancer, esophageal cancer, prostate cancer, and breast cancer.

In certain embodiments, the cancer has a prometastatic phenotype, including melanoma or breast cancer.

In certain embodiments, the cancer is a metastatic cancer. In certain embodiment, the nanoparticle disclosed herein can trigger adaptive anti-tumor activity and/or inhibit metastasis.

In certain embodiments, the subject is human, but not limited to humans, non-human primates, rodents, farm animals, canine and feline, which is to be the recipient of a particular treatment.

In certain embodiments, the method further comprises administration of an additional therapy. In certain embodiments, the method comprises administration of the anti-PD-1-CDDP-nanoparticles in combination with a therapeutically effective amount of a second agent, which is an anti-cancer agent other than the first agent.

In certain embodiments, prior to the treatment, the subject exhibits primary resistance, developed adaptive resistance and/or unresponsive to chemotherapy.

In certain embodiments, the treatment boosted an immune response and/or reduced tumor volume by at least 8-fold as compared with systemic injection of anti-PD-1. In certain embodiments, the treatment is at least 8-fold higher than administration of either anti-PD-1 or cisplatin alone. In certain embodiments, the treatment is 8-10 fold, 10-12 fold, 12-15 fold, 15-20 fold higher.

In certain embodiments, the effective dose of anti-PD-1-cisplatin is 100 μM-150 μM, 150 μM-200 μM, 200 μM-250 μM, 250 μM-300 μM, 300 μM-350 μM, 350 μM-400 μM, 400 μM-450 μM, 450 μM-500 μM, 500 μM-550 μM, 550 μM-600 μM, or 600 μM-650 μM.

In certain embodiments, the treatment inhibits cell proliferation and/or enhance T-cell infiltration.

### 4. BRIEF DESCRIPTION OF THE FIGURES

The patent or application file contains at least one drawing executed in color. Copies of this patent or patent application publication with color drawings will be provided by the Office upon request and payment of the necessary fee.

FIGS. 1A-F. Characterization of aPD-1/CDDP@NPs and MNs. (A) aPD-1/CDDP@NPs exhibited spherical morphology with a homogenous distribution, as illustrated by TEM images. (B) Particle size detected through DLS. (C) aPD-1 release profile from the NPs in PBS with diverse pH values. (D) SEM images of dissolving MNs. (E) Higher magnification of the MN tips. (F) Fluorescence microscopy image of the MNs. The NPs were labeled using Liss Rhod PE lipids. The error bar was the standard deviation (SD) of the samples (n=3).

FIGS. 2A-D. In vitro antitumor profiles and illustration of PD-L1 expression. (A) IC50 of the three cell lines (FaDu, CAL 27 and SCC-VII) treated with three drugs (CDDP, CDDP@NPs, and aPD1/CDDP@NPs). (B) Cellular uptake of the cell lines after being treated with Pt concentration of 100 μM in a 24-well plate for 4 h. (C) Cells were treated with free CDDP or aPD-1-CDDP-NPs with the Pt concentration of 100 μM for 4 h. Cell apoptosis and cell cycle were determined using the APO-BrdUTM TUNEL Assay Kit, and they both were evaluated through flow cytometry. (D) Cell cycle was evaluated based on the florescence intensity of PI. Both SCC VII cell line and RAW 264.7 cell line were treated with or without LPS. The error bar represents the SD of three independent experiments, statistical significance was calculated through one-way ANOVA. P value: ***p<0.01; **** p<0.0001.

FIGS. 3A-C. Antitumor efficacy in vivo. (A) Representative bioluminescent images of mice with various treatments: (i) control group treated with PBS (indicated as PBS); (ii) intraperitoneal (i.p.) injection of CDDP (indicated as CDDP); (iii) i.p. injection of aPD-1 (indicated as aPD-1); (iv) i.p. injection of both aPD1 and CDDP (indicated as aPD-1+CDDP); (v) i.p. injection of aPD-1-CDDP-NPs (indicated as aPD1-CDDP-NPs); (vi) CDDP NPs loaded with MN patches (indicated as CDDP-NPs MN); (vii) aPD-1 loaded MN patches (indicated as aPD-1 MN); (viii) aPD-1-CDDP-NPs loaded MN patches (indicated as aPD-1-CDDP-NPs MN) at different time points (T1: 3 days after 1st treatment, T2: 3 days after 2nd treatment, and T3: 3 days after 3rd treatment). Antitumor efficiency was evaluated based on the (B) tumor volume and (C) tumor weight after sacrifice. Each groups [(i) PBS; (ii) aPD-1; (iii) aPD1+CDDP; (iv) aPD-1/CDDP@NPs; (v) aPD-1 MNs; (vi) aPD-1/CDDP@NPs MNs] started treatment once the tumor volume reached at 10 mm3. Three treatments were given for each groups and lasting for three cycles. The error bar represents the SD for each groups (n=7). Statistical analysis was performed using the Mann-Whitney U tests. P value: *p<0.05, ***p<0.001.

FIGS. 4A-D. Tumour-specific cell proliferation and induced cell apoptosis. (A) Representative Ki-67 IHC in tumor tissues in various groups. (B) TUNEL images in tumor tissues. (C) Results of the statistical analysis. Ki-67 density was presented as the positive area/total area. (D) The apoptotic index was calculated as green cell number/total cell number. Each bar represents the mean ±SD, n=7. Statistical analysis was performed using Mann-Whitney U tests. P value: *p<0.05, **p<0.01.

FIGS. 5A-E. T-cell responses and immune profile. (A) Representative images of CD4+ T-cells and CD8+ T-cells infiltration in tumor tissues detected using flow cytometry. (B) IFN-γ detected using ELISA in serum. (C) Quantitative analysis of CD8+ T-cells among tumor cells. (D) Quantitative analysis of CD4+ T-cells among tumor cells. (E) The percentages of CD4+ FOXP3+ Tregs within total CD4+ TILs. Each bar represents the mean ±SD, n=7. Statistical analysis was performed using Mann-Whitney U tests. P value: *p<0.05, **p<0.01.

FIGS. 6A-D. Systemic effects in each group. (A) Body weight during the whole experiment. (B) BUN value and (C) total IgG value in serum. The error bars were based on the SD of 7 mice. (D) H&E staining of main organs (liver, lung, kidney and spleen) in six groups (control, aPD-1, aPD-1+CDDP, aPD-1/CDDP@NPs, aPD-1 MNs, and aPD-1/CDDP@NPs MNs). The CDDP groups exhibited severe side effects, which could be reduced through nano-encapsulation and MN-mediated delivery. Each bar represents the mean ±SD of seven mice. Statistical analysis was performed using the Mann-Whitney U tests. P value: *p<0.05, ***p<0.001.

FIG. 7. Schematic illustration of synergistic effects of immuno-chemotherapy delivered by microneedle. aPD-1 and CDDP were encapsulated into NPs for the combination of chemotherapy and immunotherapy. Then the NPs were embedded into the MN for transdermal delivery. aPD-1 could competitively block the binding of PD-L1 to PD-1, leading to activation of T-cells. Meanwhile, intracellular release of CDDP could induce direct cytotoxicity to the tumor cells.

FIGS. 8(A)-(C). (A) The PD-L1 protein expression determined through western blot. (B) mRNA expression evaluated using a TaqMan qPCR probe. (C) Representative micrographs of PD-L1 expression detected through IHC on tumor sections. Negative PD-L1 expression (partial or complete cell membrane staining less than 1%); low PD-L1 expression (approximately 50% of membranes were stained); high PD-L1 expression (over 50% of cell membranes were stained).

FIG. 9. Antitumor efficacy in vivo. Representative bioluminescent images of mice with various treatments CDDP and CDDP@NPs MNs at different time points (T1: 3 days after 1st treatment, T2: 3 days after 2nd treatment, and T3: 3 days after 3rd treatment).

FIG. 10. Systemic effects. H&E staining of main organs (liver, lung, kidney and spleen) in CDDP and CDDP@NPs MNs.

### 5. DETAILED DESCRIPTION

### 5.1 Transdermal System

The present disclosure provides a transdermal delivery device including microneedles for delivery of nanoparticles comprising immunotherapeutic agent and chemotherapeutic agent.

The transdermal delivery device may be in the form of a patch that may include various features. For example, the device may include a reservoir, e.g., a vessel, a porous matrix, etc., that may store and agent and provide the agent for delivery. The device may include a reservoir within the device itself. For instance, the device may include a hollow, or multiple pores that may carry one or more agents for delivery.

The therapeutic agent may be released from the device via degradation of a portion or the entire device or via diffusion of the agent from the device. A formulation including one or more agents may be retained within a reservoir. Materials suitable for use as impermeable backing layer can include materials such as polyesters, polyethylene, polypropylene and other synthetic polymers. The material is generally heat or otherwise sealable to the backing layer to provide a barrier to transverse flow of reservoir contents.

### 5.2 Microneedles

Microneedles (MN) are used in a wide variety of medical and scientific applications. Microneedle is a minimally invasive device that can penetrate the stratum corneum of the skin or mucosa for transdermal delivery. Skin is a highly active immune organ containing a large population of resident antigen-presenting cells. MNs piercing the immune-cell-rich epidermis can provoke robust immune responses by activating T-cells.

In one embodiment, the microneedle is formed from, but not limited to, glass, metal, plastic, or a polymeric material. Types of glass include but are not limited to borosilicate glass, with or without inner filaments, aluminosilicate glass, and quartz. Borosilicate is commonly used in applications including microinjection, patch clamp, microneedle aspiration, etc. Aluminosilicate glass may be preferred for microinjection in some cases, as it is more rigid than borosilicate glass and is capable of withstanding forces associated with microinjection. However, aluminosilicate glass is malleable at a higher temperature than borosilicate and workable over a narrower temperature range. Thus, microneedle tips made from aluminosilicate often have a shape of a fine tip with a short taper. Quartz exhibits superior mechanical, electrical, and optical qualities properties as compared to other types of glasses, due to its purity, but may be more expensive. Any of the aforementioned types of glass are suitable for use with the techniques provided herein. Other types of glass include but are not limited to soda-lime glass, neutral glass, aluminum silicate glass, lead glass, UV-glass, X-ray glass, sealing glass, etc. Other materials, such as metal, may also be used to form microtubes/microneedles. Metals include but are not limited to stainless steel, titanium alloy, copper, aluminum, chrome, shape memory alloy, nitinol, platinum, or nickel. In these embodiments, the metal may be anodized. In this case, suitable reagents for non-adhesion and color marking should be capable of or modified to be capable of binding to the surface of the metal tip. Types of plastic include but are not limited to molded plastics and/or plastics generated from the following: High-Density Polyethylene (HDPE), Polyvinyl Chloride (PVC), Low-Density Polyethylene (LDPE), Polypropylene (PP), Polystyrene or Styrofoam (PS), and Miscellaneous plastics (including polycarbonate, polylactide, acrylic, acrylonitrile butadiene, styrene, fiberglass, and nylon).

Polymeric materials may also be used for additive manufacturing and to form microneedles. Polymeric materials may include but are not limited to polyglycolic acid (PGA) or polycolic acid (PCA), polylactic acid or polylactide (PLA), acrylate, or any other type of material suitable for 3D fabrication or micro-printing techniques or additive manufacturing techniques. PLA is a biodegradable and bioactive thermoplastic aliphatic polyester that is often used in implantable medical devices due to having a high degree of biocompatibility with humans, as the degradation product, lactic acid, is metabolically innocuous. PGA is also a biodegradable, thermoplastic, linear, aliphatic polyester, which may be used to form the microneedles described herein, and is often used in biomedicine and tissue-engineering applications. Any material suitable for 3D printing applications, such as an acrylate group type material, may be used with the devices and techniques provide herein.

In certain embodiments, 3D printing may be used to form microneedles, and once formed, the microneedles may be coated with a tint/color and a non-adhesive coating. In certain embodiments, the tint/color may be mixed with the liquid polymer and incorporated into the printed microneedle during manufacturing. In certain embodiments, the microneedle can be formed from different materials such that one of the materials is colored, such as in multi-material DLW, or other additive manufacturing technologies. In general, the materials are biocompatible.

In certain embodiments, the microneedles may include any suitable shape. For example, the tip of the microneedle may be beveled, pointed, blunt, rounded, curved, or otherwise shaped. In some cases, openings may be present in the sides of the microneedle tips. In certain embodiments, the capillary tube has an outside diameter ranging from 0.5 mm to 2.00 mm prior to pulling with a pipette puller. The microneedles formed from the capillary tubes may have an inside diameter ranging from 0.2 μm to 1.56 mm, or any diameter in between, as customized based on the experimental application. In certain embodiments, microneedles may have an outer diameter between 65-180 μm and an inner diameter of 5-30 μm. Many shapes and sizes are possible, and all such shapes and sizes fall within the scope of the embodiments provided herein.

In certain embodiments, the dimensions of the microneedle may be sized based upon the material from which it is formed, the material to be injected or transferred through the microneedle tip, and/or the material into which the microneedle is inserted. Accordingly, the microneedle may be of any suitable length with any suitable diameter. Microneedle inner walls can include microstructures or anti-adhesive materials/coatings to prevent adhesion of a cell or other material being injected into an object to the inner walls of the microneedle. Microneedle outer walls can include anti-clogging materials/coatings to prevent clogging of the microneedle tip by cytoplasmic or other components during injection when the microneedle is inside the biological object.

In certain embodiments, the nanoparticle-releasing soluble microneedle structure consisting of a biocompatible amphiphilic block copolymer, which contains a water- or fat-soluble drug for easy delivery thereof. In one embodiment, the biocompatible amphiphilic block copolymer may be a di-block, tri-block or multi-block copolymer of a polymer in a hydrophilic domain and a polymer in a hydrophobic domain. In certain embodiments, the polymer in a hydrophilic domain may be one or more selected from the group consisting of polyacrylic acid (PAA), polyethylene glycol (PEG), polyacrylonitrile (PAN), polyethylene oxide (PEO), polyvinyl acetate (PVAc), polyvinyl alcohol (PVA), and polymethylmethacrylate (PMMA).

In certain embodiments, the polymer in a hydrophobic domain may be one or more selected from the group consisting of polypropylene oxide (PPO), polycaprolactone (PCL), polylactic acid (PLA), polyglycolic acid (PGA), poly(lactic-co-glycolic acid) (PLGA), polyanhydride, polyorthoester, polyester, polyesteramide, polystyrene, polydiene, polyisobutylene, polyisopropylacrylamide, polysiloxane, poly(2-vinyl naphthalene), poly(vinyl pyridine and N-methyl vinyl pyridinium iodide)), and poly(vinyl pyrrolidone).

In certain embodiments, the biocompatible amphiphilic block copolymer is preferably one or more selected from the group consisting of a poloxamer (polyethylene oxide-polypropylene oxide-polyethylene oxide) (PEO-PPO-PEO)) tri-block copolymer, a poloxamer (polypropylene oxide-polyethylene oxide-polypropylene oxide) (PPO-PEO-PPO)) tri-block copolymer, a polyethylene oxide-polylactic acid-polyethylene oxide (PEO-PLA-PEO) tri-block copolymer, a polylactic acid-polyethylene oxide-polylactic acid (PLA-PEO-PLA) tri-block copolymer, a polyethylene oxide-polyglycolic acid-polyethylene oxide (PEO-PGA-PEO) tri-block copolymer, a polyglycolic acid-polyethylene oxide-polyglycolic acid (PGA-PEO-PGA) tri-block copolymer, a polyethylene oxide-poly(lactic-co-glycolic acid)-polyethylene oxide (PEO-PLGA-PEO) tri-block copolymer, a poly(lactic-co-glycolic acid)-polyethylene oxide-poly(lactic-co-glycolic acid) (PLGA-PEO-PLGA) tri-block copolymer, a polyethylene oxide-polycaprolactone-polyethylene oxide (PEO-PCL-PEO) tri-block copolymer, a polycaprolactone-polyethylene oxide-polycaprolactone (PCL-PEO-PCL) tri-block copolymer, a polyethylene oxide-polylactic acid (PEO-PLA) di-block copolymer, a polyethylene oxide-polyglycolic acid (PEO-PGA) di-block copolymer, a polyethylene oxide-poly(lactic-co-glycolic acid) (PEO-PLGA) di-block copolymer, and a polyethylene oxide-polycaprolactone (PEO-PCL) di-block copolymer.

In certain embodiments, the biocompatible amphiphilic block copolymer is more preferably a poloxamer (polyethylene oxide-polypropylene oxide-polyethylene oxide) (PEO-PPO-PEO) tri-block copolymer.

In certain embodiments, the microneedles comprise nanoparticles that comprise one or more therapeutics. There is no particular limitation to the contained drug, and either a water-soluble or fat-soluble drug may be used. Examples of available drugs may include one selected from the group consisting of a chemical substance, an adjuvant, a vaccine, a protein drug, a peptide drug, a nucleic acid molecule for gene therapy, an active material for a cosmetic, and an antibody for medical use, or a mixture of two or more thereof.

In certain embodiments, the drug may be 0.0001 to 50 wt %, and preferably 0.01 to 20 wt % based on the total weight of the microneedle after drying. The content of the drug may be determined differently according to the minimal effective concentration of the drug and a type of the microneedle.

In certain embodiments, the microneedle may further include an additive which reinforces drug stability in the structure and needle strength. The additive may be one selected from the group consisting of hyaluronic acid, chitosan, polyvinyl alcohol, a carboxyvinyl polymer, an acrylvinyl polymer, dextran, carboxymethylcellulose, hydroxyethylcellulose, xanthan gum, locust bean gum, an ethylene-vinyl acetate polymer, cellulose acetate, acryl-substituted cellulose acetate, polyurethane, polycaprolactone, poly(lactic-co-glycolic acid) (PLGA), polylactic acid (PLA), polyglycolic acid (PGA), polyanhydride, polystyrene, polyvinyl acetate, polyvinyl chloride (PVC), polyvinyl fluoride (PVF), polyvinyl imidazole, a chlorosulfonate polyolefin, polyethylene oxide, polyvinylpyrrolidone (PVP), polyethylene glycol (PEG), polymethacrylate, hydroxypropylmethylcellulose (HPMC), ethylcellulose (EC), hydroxypropylcellulose (HPC), carboxymethylcellulose and cyclodextrin, or a mixture of two or more thereof.

In certain embodiments, the microneedle comprising the nanoparticles may be dissolved when inserted into the in vivo epithelium.

In certain embodiments, the microneedle maintains a stable structure in an aqueous solution and the nanoparticles dissolved in the aqueous solution, and easily deliver the therapeutics as well as increasing drug solubility in the aqueous solution during delivery of a hydrophobic drug, thereby facilitating hydrophobic drug delivery or simultaneous transdermal delivery of one or more therapeutics.

### 5.3 Nanoparticles Comprising Therapeutic Agents

Provided herein are nanoparticles comprising two or more therapeutic agents. Provided herein are liposomal nanoparticles and methods of making and using thereof. In certain embodiments, the nanoparticles contain one or more lipids. In certain embodiments, the lipids are PEG-conjugated lipids, and optionally one or more additional materials that physically and/or chemically stabilize the particles. In certain embodiments, the concentration of the lipid is from about 8 mole percent to about 30 mole percent. The liposomes can be prepared by any means known in the art. In certain embodiments, the nanoparticles have an average diameter of about 100 nm to about 300 nm, preferably from about 100 nm to about 250 nm, more preferably from about 100 nm to about 200 nm. The liposomes contain one or more therapeutic, prophylactic, and/or diagnostic agent to be delivered to a surface. In certain embodiments, the surface is a mucosal surface, including those that cover the female reproductive tract, gastrointestinal tract, lung, airway, nose, colon, and eyes. To deliver therapeutics efficiently across the mucosal surfaces, drug or gene carriers must be able to penetrate the mucus barrier to avoid the mucus-induced aggregation and rapid clearance.

The nanoparticles may be combined with one or more pharmaceutically acceptable excipients to prepare pharmaceutical formulations. The nanoparticles may be administered by a variety of routes of administration, such as enteral or parenteral, as well as topical or pulmonary. In one embodiment, the nanoparticles are dispensed via microneedles. In one embodiment, the nanoparticles are dispensed via microneedles in a transdermal system. In one embodiment, the transdermal system is a patch.

In one embodiment, the nanoparticles are prepared through reverse-phase microemulsion.

In certain embodiments, the nanoparticles comprises 1,2-dioleoyl-sn-glycerol-3-phospate (DOPA) formed a bilayer to enable linking with other lipids, such as 1,2-dioleoyl-3-trimethy-lammonium-propane(DOTAP), 1,2-distearoyl-sn-glycero-3phosphoethanolamine-N-[amino(polyethylene glycol)-2000] (ammonium salt) (DSPE-PEG-AA) and cholesterol.

In certain embodiments, the nanoparticles have a diameter of 10 to 2000 nm, and preferably 50 to 1000 nm.

The nanoparticles may also contain one or more stabilizers. Stabilizers are components or additional components in the nanoparticles that reduce or prevent vesicle destabilization and/or opsonization and concomitant release of encapsulated agents or drugs. For example, stabilizers, such as cholesterol and other materials, enhance the mechanical strength of the lipid bilayer. Other materials include one or more of the lipids. The concentration of the stabilizer(s) is at least about 5 mole percent, preferably at least about 10 mole percent, more preferably at least about 20 mole percent, most preferably at least about 30 mole percent. In some embodiments, the concentration of the stabilizer is from about 5 mole % to about 50 mole %. In certain embodiments, the concentration of the stabilizer is about 25, 50, or 70 mole percent. In one embodiment, the concentration of the stabilizer is about 25 mole percent. In certain embodiments, the stabilizer is cholesterol and is present in a concentration as described above. Other suitable stabilizers include ganglioside. In other embodiments, the stabilizer may be a PEG-conjugated lipid and thus an additional stabilizer or stabilizers is not required.

In certain embodiments, the nanoparticles are physically and chemically stable. “Physically stable”, as used herein, means that the particle size and/or polydispersity remain constant over an extended period of time. In certain embodiments, “physically stable” means the change in the average diameter of the particle is less than 10, 9, 8, 7, 6, 5, 4, 3, 2, or 1% over, 2, 4, 6, 8, 12, 16, 20, 24, 30, 36, or 48 hours. In certain embodiments, the change in the average diameter of the particles is less than 10, 9, 8, 7, 6, 5, 4, 3, or 2% after 48 hours. In certain embodiments, “physically stable” means the change in the polydispersity of the particle is less than 10, 9, 8, 7, 6, 5, 4, 3, 2, or 1% over, 2, 4, 6, 8, 12, 16, 20, 24, 30, 36, or 48 hours. In certain embodiments, the change in the polydispersity of the particles is less than 10, 9, 8, 7, 6, 5, 4, 3, 2 or 1% after 48 hours. In certain embodiments, the nanoparticles show little or no aggregation, remaining well dispersed when introduced into mucus.

### 5.4 Methods of Treatment

In one embodiment, the present disclosure provides a method of delivering a therapeutic agent to a subject, the method comprising: penetrating a stratum corneum of the subject with a microneedle having a channel in fluid communication with nanoparticles comprising one or more therapeutic agents. In one embodiment, the therapeutic agent is delivered across a dermal barrier of a subject with high bioavailability. In one embodiment, the methods are used for transdermal delivery of a therapeutic agent to the blood stream of a subject. In one embodiment, the therapeutic agent is delivered to a tumor in a subject.

The nanoparticles may be systemically introduced into the subject to be treated. As used herein, the term “systemic introduction” refers to any introduction of nanoparticles that pertains to or affects the subject as a whole such as an introduction of nanoparticles into the circulating blood of the subject. As previously described, the mechanism by which the nanoparticles accumulate in the target area may be by a passive mechanism, an active mechanism, or a combination thereof. In the passive mechanism, nanoparticles may be injected or infused into the blood stream and accumulate at the target area or tumor site through the enhanced permeability and retention (“EPR”) effect. Through this mechanism, passively targeted particles accumulate in the tumor in a region near the disrupted blood vessels. In one embodiment, the nanoparticles are introduced into a subject via a transdermal system. In one embodiment, the transdermal system is a patch. In one embodiment, the patch comprises microneedles.

Through this mechanism, passively targeted particles accumulate in the tumor in a region near the disrupted blood vessels. In certain embodiments, the nanoparticles accumulate preferentially in tumors by the enhanced permeability and retention (EPR) effect, where the leaky tumor vasculature containing wide interendothelial junctions, abundant transendothelial channels, incomplete or absent basement membranes, and dysfunctional lymphatics contribute to passive extravasation of systemically injected macromolecules and nanoparticles into tumors.

Active mechanisms for targeting the tumor site include nanoparticles comprising an antibody to a cell surface molecule, preferentially expressed by a target cell. These particles may be inserted into the blood, allowed to selectively accumulate in the target area, and selectively bind to cells in the target area which have such molecules present on their cell surface. Additionally, vascular targeting agents may be used to actively target the target site. Similarly, particles actively targeted to the tumor endothelial cells will accumulate at an endothelial surface.

The nanoparticles described herein exhibit enhanced transport when administered a subject. In one embodiment, the nanoparticles are administered via microneedles on a transdermal system. In some embodiments, the nanoparticles travel through the surface of a subject's body, at certain absolute diffusivities. In certain embodiments, the surface is proximal or close to a treatment area such as a tumor or cancer. In certain embodiments, the particles may travel at diffusivities of at least 1×10⁴, 2×10⁴, 5×10⁴, 1×10³, 2×10³, 5×10³, 1×10², 2×10², 4×10², 5×10², 6×10², 8×10², 1×10, 2×10, 5×10, 2, 1, 0.9, 0.8, 0.7, 0.6, 0.5, 0.4, 0.3, 0.2, or 0.1 μm²/s at a time scale of 1 s. In contrast, non-penetrating particles have a diffusivity of at least about 1×10⁴ μm²/s.

The ability of the nanoparticles to diffuse through the surface of a subject's body can also be evaluated qualitatively by visual inspection. In some embodiments, the concentration of lipid is about 10 mole percent and at least 50, 60, 70, 80, or 90% of the nanoparticles are mobile at 2 hours and at least 30, 40, 50, 60, or 70% of the nanoparticles are mobile at 15 hours. In one embodiment, the nanoparticles exhibit little or no aggregation. In other embodiments, the concentration of lipid is about 20% and at least about 75, 80, 85, 90, 95, 96, 96, 98, or 99% of the nanoparticles are mobile at 2 hours and at least about 80, 85, 86, 87, 88, 89, 90, 91, 92, 93, 94, 95, 96, 97, 98, or 99% of the nanoparticles are mobile at 15 hours. The nanoparticles are well dispersed with little or no aggregation.

By utilization of the transdermal delivery method described herein, the comparative bioavailability as compared to a subcutaneous delivery route can be greater than about 20%, greater than about 30%, or greater than about 35%.

In conjunction with high bioavailability, the concentration of a bioactive agent lost from circulation, e.g., sequestered in an organ of the body, and particularly organs associated with defense mechanisms is low. In certain embodiments, the concentration of a therapeutic agent found in lymph nodes can be less than about 50 nanograms per gram of tissue (ng/g), less than about 40 ng/g, less than about 20 ng/g, less than about 10 ng/g, less than about 1 ng/g, or less than about 0.6 ng/g following delivery of the agent, for instance about 72 hours following delivery. In certain embodiments, the concentration of the therapeutic agent within a tumor or a target organ for treatment is more than about 40-50 ng/g, more than about 25-40 ng/g, or more than about 15-25 ng/g.

Cancer cells are unlike normal tissue cells in that regular cellular mechanisms and behaviors are absent making choice of therapy and resulting efficacy less predictable. In particular, a subject may exhibit resistance, developed adaptive resistance and/or unresponsive to therapy. Drug resistance is an issue and some cancer cells may survive chemotherapy and immunotherapy because they are resistant to the cancer drug. Cancer drug resistance may be due to increased cancer drug metabolism by the cancer cell or by an increased rate of cancer drug transport out of the cancer cell by a cancer drug membrane transporter, so that the intracellular cancer drug concentration remains sub-toxic to the cancer cell.

In one embodiment, the methods provide herein comprises administration of nanoparticles to enhance immunotherapy and chemotherapy-induced cancer cell death and reduce the toxicity associated with immunotherapy and chemotherapy. In one embodiment, administration of microneedles comprising nanoparticles comprising immunotherapeutic agent and a chemotherapeutic agent provides a synergistic effect as compared to when the immunotherapeutic agent and chemotherapeutic agent are administered alone. In one embodiment, the nanoparticles comprise both an antibody and cisplatin which provides synergy or synergistic effects, i.e., the effect achieved when the active ingredients used together is greater than the sum of the effects that results from using the compounds separately. Additional combination therapy may be administered which could provide further synergistic effects.

In certain embodiments, the method provided herein is used to promote a positive therapeutic response with respect to cancer. The term “positive therapeutic response” with respect to cancer treatment refers to an improvement in the disease in association with the activity of the disclosed nanoparticles comprising, e.g., antibodies or antigen-binding fragments, variants, or derivatives thereof, and/or an improvement in the symptoms associated with the disease. Thus, for example, an improvement in the disease can be characterized as a complete response. By “complete response” is intended an absence of clinically detectable disease with normalization of any previously test results. Alternatively, an improvement in the disease can be categorized as being a partial response. A “positive therapeutic response” encompasses a reduction or inhibition of the progression and/or duration of cancer, the reduction or amelioration of the severity of cancer, and/or the amelioration of one or more symptoms thereof resulting from the administration of the disclosed nanoparticles.

In certain embodiments, the treatment provides one, two or three or more results following the administration of the nanoparticles disclosed herein: (1) a stabilization, reduction or elimination of the cancer cell population; (2) a stabilization or reduction in cancer growth; (3) an impairment in the formation of cancer; (4) eradication, removal, or control of primary, regional and/or metastatic cancer; (5) a reduction in mortality; (6) an increase in disease-free, relapse-free, progression-free, and/or overall survival, duration, or rate; (7) an increase in the response rate, the durability of response, or number of patients who respond or are in remission; (8) a decrease in hospitalization rate, (9) a decrease in hospitalization lengths, (10) the size of the cancer is maintained and does not increase or increases by less than 10%, preferably less than 5%, preferably less than 4%, preferably less than 2%, and (12) an increase in the number of patients in remission.

Clinical response can be assessed using screening techniques such as magnetic resonance imaging (MRI) scan, x-radiographic imaging, computed tomographic (CT) scan, flow cytometry or fluorescence-activated cell sorter (FACS) analysis, histology, gross pathology, and blood chemistry, including but not limited to changes detectable by ELISA, RIA, or chromatography.

Also provided herein is a combination therapy comprising administration of nanoparticles comprising an immunotherapeutic agent and a chemotherapeutic agent and further administration of an additional therapy. The methods disclosed herein encompass co-administration, using separate formulations or a single pharmaceutical formulation, and consecutive administration in either order. In certain embodiments, the nanoparticles are administered in combination with other drugs, wherein the antibody or antigen binding fragment, variant, or derivative thereof and the therapeutic agent(s) can be administered sequentially, in either order, or simultaneously (i.e., concurrently or within the same time frame).

### 5.5 Therapeutic Agents

Anticancer agents include drugs used to treat malignancies, such as cancerous growths. Drug therapy can be used alone, or in combination with other treatments such as surgery or radiation therapy. Several classes of drugs can be used in cancer treatment, depending on the nature of the organ involved. For example, breast cancers are commonly stimulated by estrogens, and can be treated with drugs which inactive the sex hormones. Similarly, prostate cancer can be treated with drugs that inactivate androgens, the male sex hormone. Anti-cancer agents for use in certain methods of the present disclosure include, among others, antibodies (e.g., antibodies which bind IGF-1R, antibodies which bind EGFR, antibodies which bind Her2, or antibodies which bind cMET), small molecules targeting IGF1R, small molecules targeting EGFR, small molecules targeting Her2, antimetabolites, alkylating agents, topoisomerase inhibitors, microtubule targeting agents, kinase inhibitors, protein synthesis inhibitors, immunotherapeutic agents, hormonal therapies, glucocorticoids, aromatase inhibitors, mTOR inhibitors, chemotherapeutic agents, Protein Kinase B inhibitors, Phosphatidylinositol 3-Kinase (PI3K) inhibitors, Cyclin Dependent Kinase (CDK) inhibitors, RLr9, CD289, enzyme inhibitors, anti-TRAIL, MEK inhibitors, etc.

In certain embodiments, the nanoparticles disclosed herein comprises antibodies or antigen-binding fragments thereof, against PD-1 (programmed death 1 protein), its two ligands PD-L1 (programmed death ligand 1) and/or PD-L2, or CTLA-4 (cytotoxic T lymphocyte antigen 4 protein). In certain embodiments, the anti-PD-1 antibody is pembrolizumab (KEYTRUDA.RTM., formerly lambrolizumab, also known as MK-3475) or an antigen-binding fragment thereof. In certain embodiments, the anti-PD-1 antibody is nivolumab (BMS-936558, MDX-1106, ONO-4538, OPDIVA.RTM.) or an antigen-binding fragment thereof. In certain embodiments, the anti-PD-L1 antibody is BMS-936559 or an antigen binding fragment thereof. In certain embodiments, the anti-PD-L1 antibody is MPDL3280A. In certain embodiments, the anti-PD-1 antibody is AMP-224 (anti-PD-1 Fc fusion protein) or an antigen binding fragment thereof. In certain embodiments, the anti-PD-L1 antibody is MEDI4736 or an antigen binding fragment thereof.

In one embodiment, chemotherapeutic agent is selected from the group consisting of sorafenb, regorafenib, imatinib, eribulin, gemcitabine, capecitabine, 22 elphalan, lapatinib, dabrafenib, sutinib malate, crizotinib, everolimus, torisirolimus, sirolimus, axitinib, gefitinib, anastrole, bicalutamide, fulvestrant, ralitrexed, pemetrexed, goserilin acetate, erlotininb, vemurafenib, visiodegib, tamoxifen citrate, paclitaxel, docetaxel, cabazitaxel, oxaliplatin, ziv-aflibercept, bevacizumab, trastuzumab, pertuzumab, 22 elphalan 22 b, taxane, bleomycin, 22 elphalan, plumbagin, camptosar, mitomycin-C, mitoxantrone, SMANCS, doxorubicin, pegylated doxorubicin, Folfori, 5-fluorouracil, temozolomide, pasireotide, tegafur, gimeracil, oteraci, itraconazole, bortezomib, lenalidomide, irintotecan, epirubicin, and romidepsin. In certain embodiments, the chemotherapeutic agents are Cisplatin, Carboplatin, Fluorouracil, Vinblastine, Gemcitabine, Cyclophosphamide, Doxorubicin, Methotrexate, Paclitaxel, Topotecan, Etoposide, Methotrexate, Sorafenib, Irinotecan, Tarceva or a combination thereof.

### 5.6 Mode of Administration

Methods of preparing and administering nanoparticles comprising antibodies, or antigen-binding fragments, variants, or derivatives thereof and cisplatin to a subject in need thereof are well known to or are readily determined by those skilled in the art. In certain embodiments, the nanoparticles are provided in the form of a composition comprising the nanoparticles and a pharmaceutical carrier. In certain embodiments, the composition is a topical composition comprising nanoparticles, a surfactant, an oil and water. In certain embodiments, the nanoparticle composition is a micro-emulsion. The route of administration can be, for example, oral, parenteral, by inhalation or topical. The term parenteral as used herein includes, e.g., intravenous, intraarterial, intraperitoneal, intramuscular, subcutaneous, rectal, or vaginal administration. However, in other methods compatible with the teachings herein, the nanoparticles can be delivered directly to the site of the adverse cellular population thereby increasing the exposure of the diseased tissue to the therapeutic agent. In one embodiment, the nanoparticles are delivered via microneedles.

The route of administration of the combination thereof can be, for example, oral, parenteral, by inhalation or topical. The term parenteral as used herein includes, e.g., intravenous, intraarterial, intraperitoneal, intramuscular, subcutaneous, rectal, or vaginal administration. However, in other methods compatible with the teachings herein, a combination of the present disclosure can be delivered directly to the site of the adverse cellular population thereby increasing the exposure of the diseased tissue to the therapeutic agent.

### 5.7 Pharmaceutical Composition

The pharmaceutical compositions used in this disclosure can comprise pharmaceutically acceptable carriers, including, e.g., water, ion exchangers, proteins, buffer substances, and salts. Preservatives and other additives can also be present. The carrier can be a solvent or dispersion medium. Suitable formulations for use in therapeutic methods disclosed herein are described in Remington's Pharmaceutical Sciences (Mack Publishing Co.) 16^{th} ed. (1980). In any case, sterile injectable solutions can be prepared by incorporating a therapeutic combination of the invention an active compound (e.g., an antibody, or antigen-binding fragment, variant, or derivative thereof with a chemotherapeutic drug, in combination with other active agents) in the required amount in an appropriate solvent followed by filtered sterilization. Further, the preparations can be packaged and sold in the form of a kit. Such articles of manufacture can have labels or package inserts indicating that the associated compositions are useful for treating a subject suffering from, or predisposed to a disease or disorder.

Parenteral formulations can be a single bolus dose, an infusion or a loading bolus dose followed with a maintenance dose. These compositions can be administered at specific fixed or variable intervals, e.g., once a day, or on an “as needed” basis. The composition can be administered as a single dose, multiple doses or over an established period of time in an infusion. Dosage regimens also can be adjusted to provide the optimum desired response (e.g., a therapeutic or prophylactic response).

Therapeutically effective doses of the compositions of the present disclosure for treatment of disorder such as certain types of cancers including e.g., colon cancer, melanoma, breast cancer, lymphoma, non-small cell lung carcinoma Hodgkin's lymphoma, non-Hodgkin's lymphoma, and Burkitt's lymphoma, ovarian cancer, breast cancer, head and neck cancers, and pancreatic cancer, vary depending upon many different factors, including means of administration, target site, physiological state of the patient, whether the patient is human or an animal, other medications administered, and whether treatment is prophylactic or therapeutic. Usually, the patient is a human, but non-human mammals including transgenic mammals can also be treated. Treatment dosages can be titrated using routine methods known to those of skill in the art to optimize safety and efficacy.

### 5.8 Dosages

In certain embodiments, the nanoparticles is administered at a concentration of about 1 mg/kg, about 2 mg/kg, about 3 mg/kg, about 4 mg/kg, about 5 mg/kg, about 6 mg/kg, about 7 mg/kg, about 8 mg/kg, about 9 mg/kg, about 10 mg/kg, about 11 mg/kg, about 12 mg/kg, about 13 mg/kg, about 14 mg/kg, about 15 mg/kg, about 16 mg/kg, about 17 mg/kg, about 18 mg/kg, about 19 mg/kg, or about 20 mg/kg. In certain embodiment, the antibody or antigen binding fragment thereof and cisplatin are administered at a ratio of about 1:1, 1:2, 1:3, 1:4, 1:5, 1:10, 10:1, 5:1, 4:1, 3:1, 2:1 or 1:1. In certain embodiments, the administration of the treatment disclosed herein can increase survival by about 10%, about 20%, about 30%, about 40%, about 50%, about 60%, about 70%, about 80%, about 90%, or about 100% compared to untreated subjects or subjects treated with a monotherapy. In certain embodiments, the administration of nanoparticles disclosed herein can increase survival by about 2-fold, about 3-fold, about 4-fold, about 5-fold, about 6-fold, about 7-fold, about 8-fold, about 9-fold, or about 10-fold compared to untreated subjects or subjects treated with a monotherapy.

In certain embodiments, the effective dose of the nanoparticles is between about 1 nanogram per kilogram patient body weight to about 50 milligrams per kilogram patient body weight; or between about 1 nanogram per kilogram patient body weight to about 5 milligrams per kilogram patient body weight; or between about 1 nanogram per kilogram patient body weight to about 0.5 milligrams per kilogram patient body weight; or between about 10 nanogram per kilogram patient body weight to about 0.5 milligrams per kilogram patient body weight; or between about 20 nanogram per kilogram patient body weight to about 100 micrograms per kilogram patient body weight; or between about 10 nanogram per kilogram patient body weight to about 10 micrograms per kilogram patient body weight. In certain embodiments, the nanoparticles in the blood plasma of the patient following administration is between about 5 nanomolar to about 200 micromolar; or between about 10 nanomolar to about 100 micromolar; or between about 20 nanomolar to about 10 micromolar.

### 6 EXAMPLES

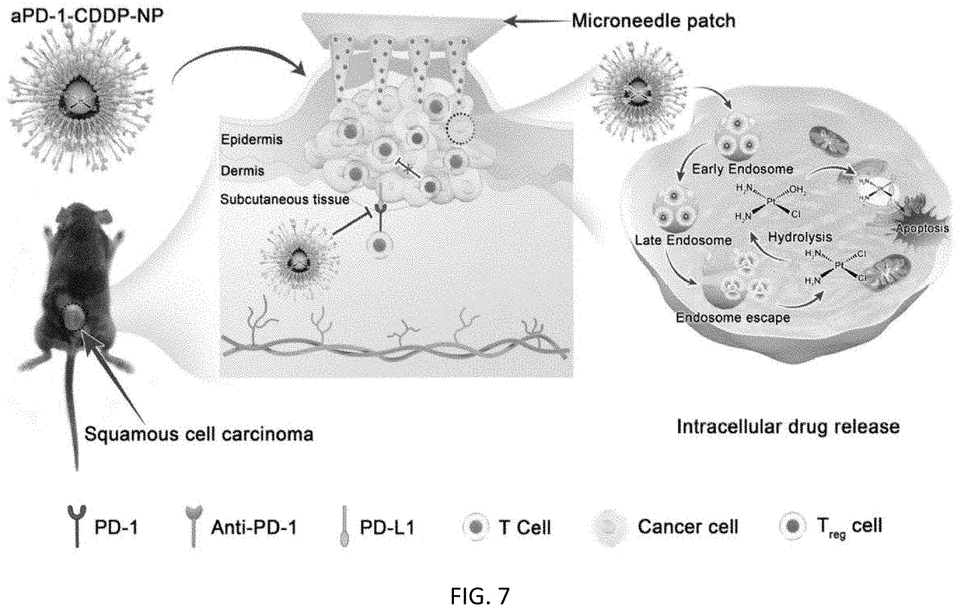

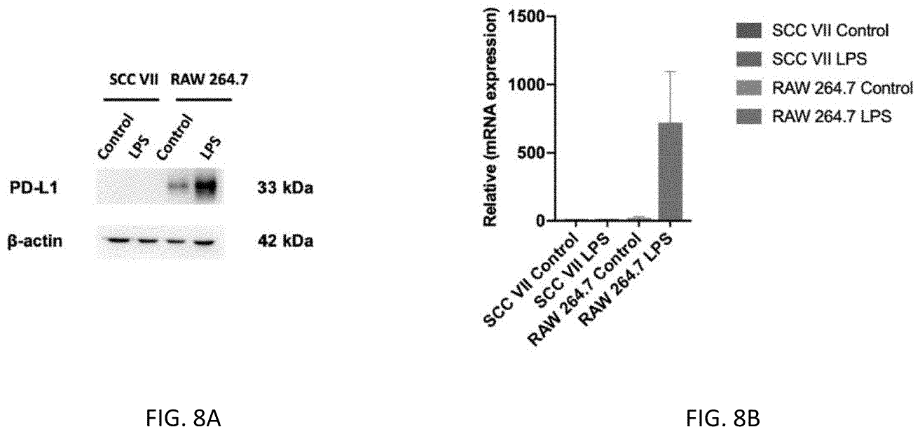

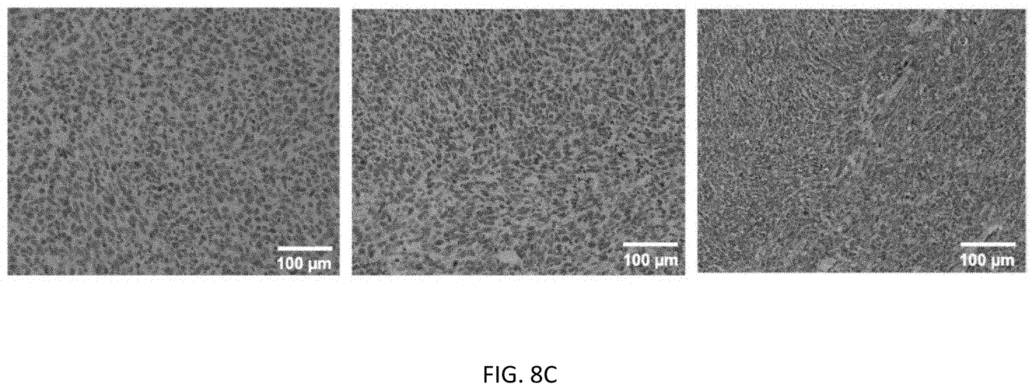

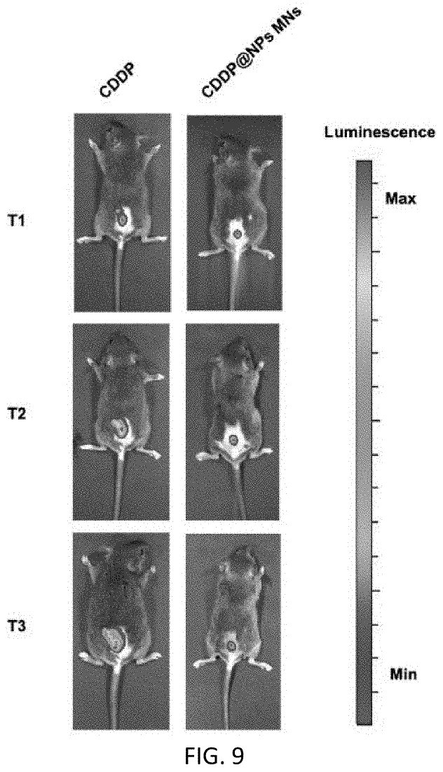

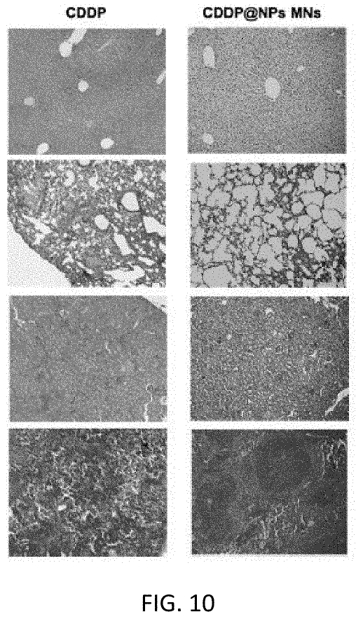

In one embodiment, disclosed herein is aPD-1 delivered by microneedles in the treatment of immunotherapy-unresponsive cancers. Also provided is a novel microneedle loaded with aPD-1-CDDP-NPs (FIG. 7) to facilitate synergistic immuno-chemotherapy. Using an immunocompetent murine tumor homograft model, we demonstrated that the robust T-cell response activated by microneedles can enhance the efficacy of both aPD-1 and CDDP. Synergistic immuno-chemotherapeutic effects were sustained through robust T-cells response activated by microneedles, blockage of PD-1 in T cells by aPD-1, and direct cytotoxicity of CDDP in tumor cells. The microneedle-mediated host immune response could augment the aPD-1-activated T-cell immunity, which in turn would enhance the direct killing of cancer cells by CDDP.

### 6.1 Characterization of aPD-1-CDDP-NPs

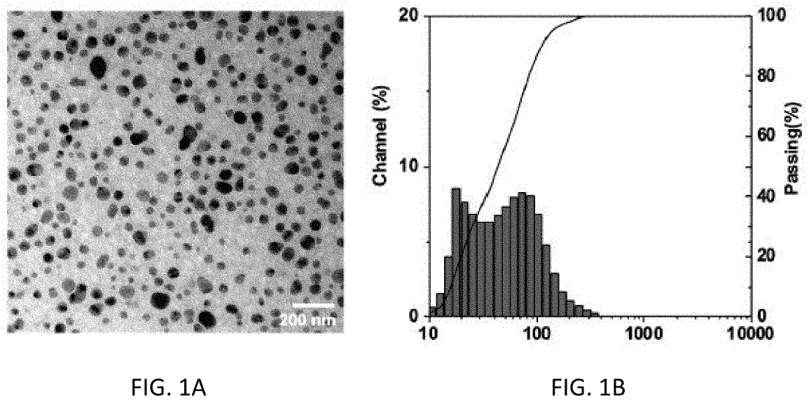

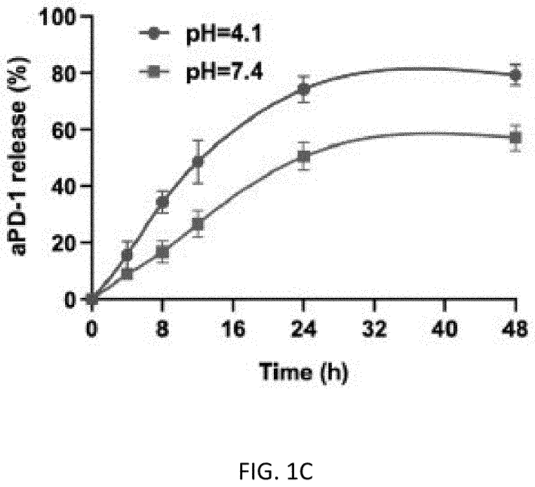

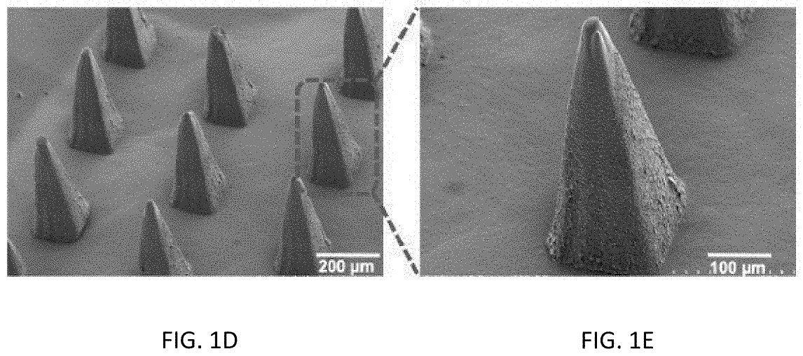

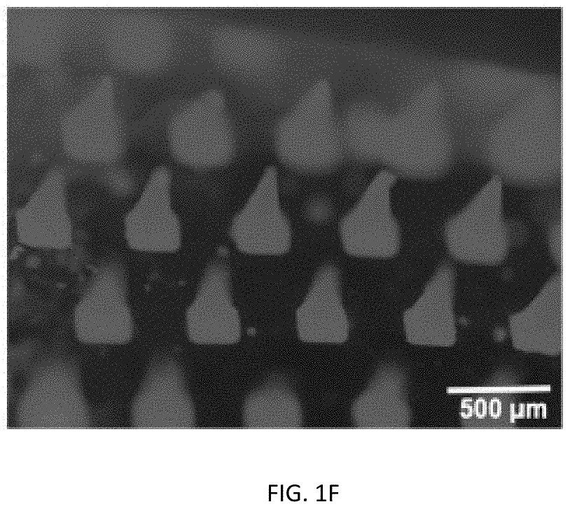

The aPD-1-CDDP-NPs were synthesized through reverse-phase microemulsion. A well-prepared CDDP precursor, cis-[Pt(NH₃)₂(H₂O)₂]₂(NO₃)₂ was utilized to increase the solubility and further facilitate the encapsulation of CDDP. 1,2-dioleoyl-sn-glycerol-3-phospate (DOPA) formed a bilayer to enable linking with other lipids, such as 1,2-dioleoyl-3-trimethy-lammonium-propane (DOTAP), 1,2-distearoyl-sn-glycero-3-phosphoethanolamine-N-[amino(polyethylene glycol)-2000](ammonium salt) (DSPE-PEG-AA) and cholesterol. These lipids present the advantages of tumor targeting and selectivity. ^{21, 22} Additionally, aPD-1 was loaded onto the outer layer of the NPs because of hydrophobic and electrostatic non-specific interactions. The encapsulation efficiency of aPD-1 was 50%, as determined using an enzyme linked immunosorbent assay (ELISA) kit. No significant antibody release was detected within one month of storage at 4° C. As depicted in transmission electron microscope (TEM) image, NPs exhibited spherical morphology with a diameter of approximately 57.6 nm (FIG. 1A), which was consistent with the result of dynamic light scattering (DLS) (FIG. 1B). The average diameter detected by DLS was 55.5 nm. To evaluate the release of aPD-1, NPs were incubated at 37° C. in PBS with various pH values. A higher proportion of aPD-1 dissociated from the NPs in acidic environment within 24 h, whereas Pt in the inner layer of the NPs could promise sustained release for 72 h (FIG. 1C). The aPD-1-CDDP-NPs facilitated the synergistic delivery of chemotherapeutic agents and immunotherapeutic agents.

### 6.2 Antitumor Activity of aPD1-CDDP-NPs In Vitro

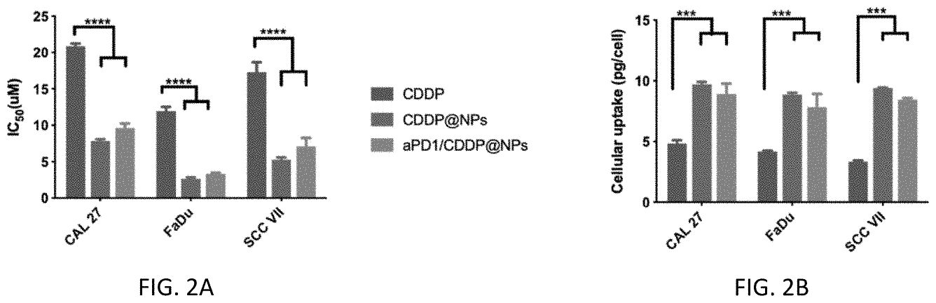

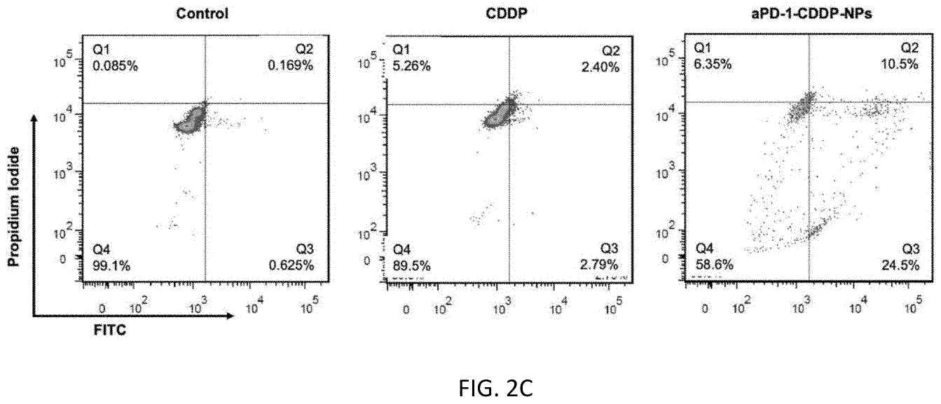

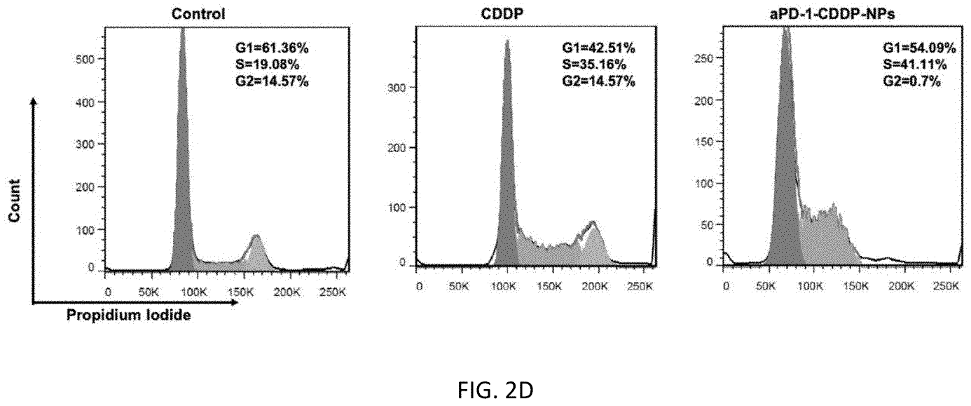

To assess the antitumor efficiency of the aPD1-CDDP-NPs, cytotoxicity, cellular uptake and cell apoptosis were detected in FaDu, CAL 27 and SCC VII cell lines. aPD1 alone could not induce any cell cytotoxicity, even when added into CDDP did not enhance its anticancer efficiency in vitro. aPD-1-CDDP-NPs were synthesized based on the procedures for synthesizing CDDP-NPs and were conferred the same advantages. The half-maximal inhibitory concentration (IC₅₀) values of NPs for the three cell lines were all significantly lower than that of the free CDDP (FIG. 2A), and more Pt was detected after the cell lines were treated with drugs at 100 μM for 4 h (FIG. 2B). In sum, more drugs were taken up by cells and they exerted higher drug efficiency after lipid coating and nano-encapsulation. NPs increased the solubility of CDDP, and their escape from endosome and avoidance of lysosome degradation due to the encapsulation of DOTAP, further increasing the drug uptake amount by the cell lines. aPD-1 was mainly used to inhibit the immune checkpoint blockade (ICB) and evoke the T-cell activity. aPD-1-CDDP-NPs showed no significant difference on both IC50 and cellular uptake compared with the CDDP-NPs, given that aPD-1 was not effective in vitro. To be concluded, aPD-1-CDDP-NPs had the same cytotoxicity for cancer cell line as CDDP-NPs in vitro.

In addition, cell lines were treated with free CDDP or aPD-1-CDDP-NPs respectively, followed with analysis through flow cytometry using APO-BrdU kit. A large number of 3hydroxyl ends appeared when DNA strands became fragmented, and they were labeled with BrdUTP and terminal deoxynucleotidyl transferase (TdT) using the TUNEL technique. After DNA incorporation, BrdU could be detected through anti-BrdU antibody. As shown in FIG. 2C, aPD-1-CDDP-NPs induced 24.5% cell apoptosis, whereas free CDDP induced only 2.79% cell apoptosis. This result indicated that NPs triggered discernable apoptosis in the cell lines. Furthermore, according to cell cycle analysis, the percentage of the G2 phase was remarkably decreased, indicating lower cell growth in the aPD-1-CDDP-NPs group (FIG. 2D).

In conclusion, lower IC50 values and higher cellular uptake have been achieved for NPs, and they could induce more apoptosis by reducing the number of cells in the G2 phase. All the data suggested that NPs could significantly enhance the antitumor efficiency, although adding aPD-1 did not internalize the medical effects.

### 6.3 Characterization of Dissolving MNs

To target the immune region on the skin through transdermal delivery, drugs were further encapsulated into dissolving MNs. Dissolving MNs were fabricated following a molding method. Polyvinylpyrrolidone (PVP) was selected as a structural material, because it offers the advantages of biocompatibility, strong mechanical property and highly water solubility. As illustrated in scanning electron microscopy (SEM) images (FIG. 1D and 1E), MN consisted of 9×9 needles with 800 μm in height and a base diameter of approximately 400 μm. Liss Rhod PE lipids were applied to label the NPs. The fluorescent image of the MNs further confirmed the adequate distribution of the therapeutic agents on MN tips (FIG. 1F). The obtained MNs could be completely dissolved in water within 5 minutes and they could be dissolved into mouse skin in 20 minutes. After the MN-array was pressed on the mouse skin with appropriate strength for 20 minutes, obvious pinholes could be observed, and tips could be dissolved in the process. Moreover, the skin region with the pinholes could recover in less than one day without causing any skin ulceration. In addition, the bioactivity of the loaded aPD-1 and NPs did not degrade after one month of storage at 4° C. Taken together, the results indicate that this type of PVP-structured MN may mediate drug delivery with a uniform distribution and high solubility in the skin, as well as mechanical strong.

### 6.4 PD-L1 Expression In Vitro and In Vivo

PD-L1 is a logical biomarker of aPD-1 and aPD-L1 therapy ^{23, 24}. The SCC VII cell line is a murine squamous cell carcinoma cell line that spontaneously arises in the abdominal wall of a C3H mouse. We determined whether PD-L1 protein and mRNA are expressed in the SCC VII cell lines through western blot and qPCR respectively. RAW 264.7 exhibited higher PD-L1 expression after LPS stimulation; however, no expression was observed in SCC VII cells (FIGS. 2F & 2J). Whether PD-L1 is expressed in an immunocompetent murine tumor homograft model was further detected using Immunohistochemistry (IHC). The representative micrographs with PD-L1 positive and negative expression are presented in FIG. 2H. Partial or complete membrane staining less than 1% is regarded as negative expression of PD-L1. In addition, a staining ratio ranging from 1% to 49% is defined as low PD-L1 expression, whereas high PD-L1 expression represents those staining ratio of more than 50% ²⁵. According to the aforementioned criteria, 85% of the tumor positively expressed PD-L1, among which 50% demonstrated high PD-L1 expression.

### 6.5 Antitumor Efficiency In Vivo

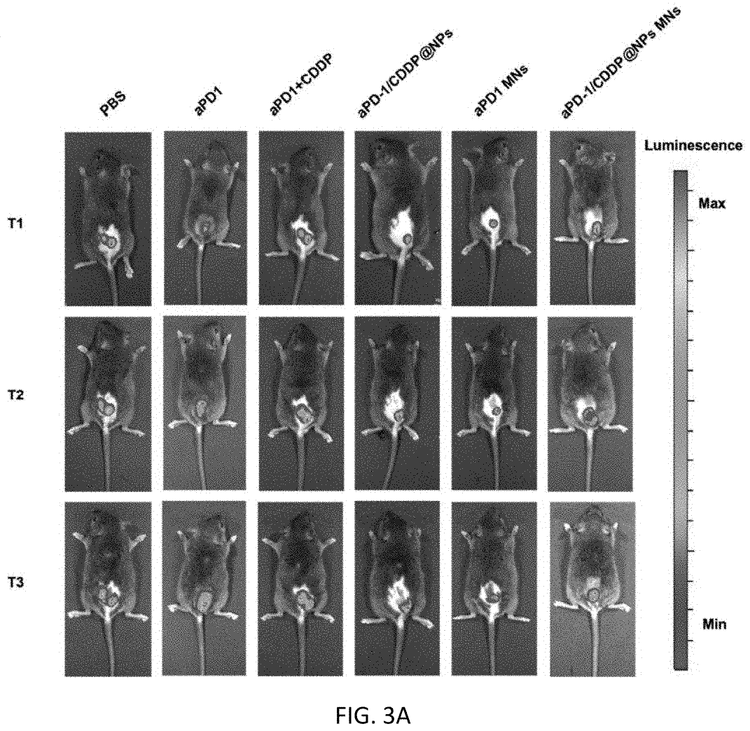

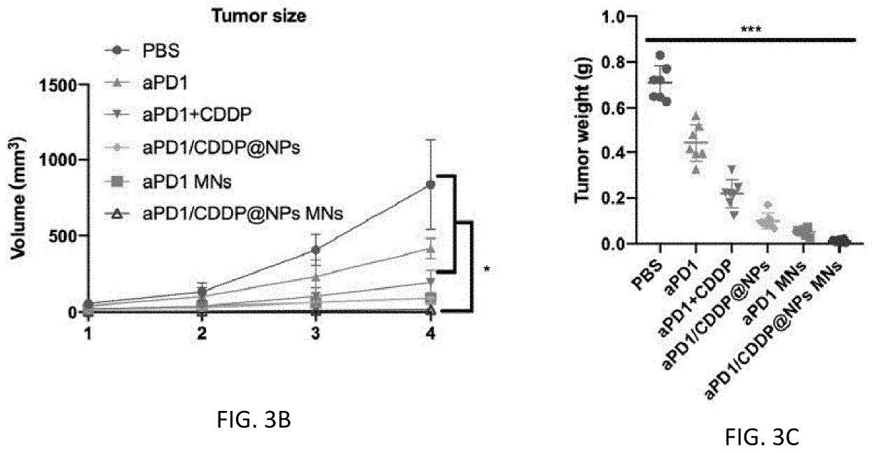

We further investigated tumor inhibition on an immunocompetent murine tumor homograft model. SCC VII cell lines were subcutaneously inoculated into the right flank of C3H/HeJ mice to obtain a superficial tumor. Mice were divided into 8 groups, including (i) control group treated with PBS (indicated as PBS); (ii) intraperitoneal (i.p.) injection of CDDP (indicated as CDDP); (iii) i.p. injection of aPD-1 (indicated as aPD-1); (iv) i.p. injection of both aPD1 and CDDP (indicated as aPD-1+CDDP); (v) i.p. injection of aPD-1-CDDP-NPs (indicated as aPD1-CDDP-NPs); (vi) CDDP NPs loaded with MN patches (indicated as CDDP-NPs MN); (vii) aPD-1 loaded MN patches (indicated as aPD-1 MN); (viii) aPD-1-CDDP-NPs loaded MN patches (indicated as aPD-1-CDDP-NPs MN). Tumor volume was monitored using a Vernier caliper before the treatment and 3 days after each treatment. Furthermore, tumor growth was double confirmed using bioluminescent images (FIG. 3A). Mice treated with PBS showed natural tumor growth. Mice treated with CDDP exhibited a limited treatment efficacy, with significantly reduced tumor weight but no difference in tumor volume compared to control group. Although 85% of tumors positively expressed PD-L1, aPD-1 systemic treatment caused a negligible inhibitory effect. aPD-1 is an immune drug that can block the PD-1/PD-L1 axis to renovate cytotoxic T cells. The ineffective therapeutic role of aPD-1, however, may be due to the following reasons. First, the renovated T cells may be too exhausted that they display impaired effector function with limited proliferative potential ²⁶. Second, cancer antigens may be “masked”; therefore, they cannot be identified by T-cells ²⁷. Third, the tumor microenvironment may contain multiple inhibitory immune cells, such as tumor-associated macrophages, which overwhelm the T-cells into have adverse effects ²⁸. Although the exact reason was unclear, the animal model in this study was unresponsive to systemic aPD-1 immunotherapy.

However, aPD-1 delivered by MN patches revealed significant anticancer effects. The tumor volume of the aPD-1 MN groups (418±66 mm³) was significantly reduced than that of the aPD-1 systemic injection group (90.252±39.343 mm³), with a p value less than 0.05. Tumor weight of the aPD-1 MN group (0.05±0.017 g) decreased by 8-fold compared with that of the aPD-1 systemic injection group (0.443±0.083 g) (p<0.001). Skin is a highly active immune organ containing a large population of resident antigen-presenting cells. It has been reported that MN was applied for increasing vaccine immunogenicity by targeting antigen delivery to skin,²⁹ and MN could induce immune responses by activating T-cells.³⁰ Our results demonstrated that aPD-1 delivered by MN could achieve potent antitumor effects in the animal model unresponsive to aPD-1 systemic therapy. This represented a promising treatment strategy for immunotherapy-unresponsive cancers.

Disclosed herein, aPD-1+CDDP group exhibited a greater tumor regression effect than either the aPD-1 or CDDP groups (p<0.05, FIG. 3). Notably, aPD-1-CDDP-NPs could synergistically deliver both chemotherapeutic and immunotherapeutic drugs to mice, and tumor growth was inhibited more significantly than in the aPD-1+CDDP groups (p<0.01, FIG. 3). Tumor weight of the aPD-1-CDDP-NPs group was 0.099±0.03 g, whereas it was 0.219±0.061 g in the aPD-1+CDDP group (p<0.01). Tumor volume in the aPD-1+CDDP group was 192.799±79.824 mm,³ while it was only 93.246±36.017 mm³ in the aPD-1-CDDP-NPs group (p<0.05). CDDP-NPs deliverer by MNs exhibited greater antitumor efficiency than aPD-1-CDDP-NPs, which confirmed the promising antitumor efficiency of MNs. Moreover, the aPD-1-CDDP-NPs MNs group displayed the most notable tumor regression effect compared with any other groups. Compared to the aPD-1-CDDP-NPs group, both tumor volume (18.312±8.286 mm³) and tumor weight (0.012±0.005 g) were significantly decreased in the aPD-1-CDDP-NPs MNs group (p<0.05 and p<0.001, respectively). These results proved that MN provided a highly promising tool for the transdermal co-delivery of chemotherapeutic and immunotherapeutic drugs.

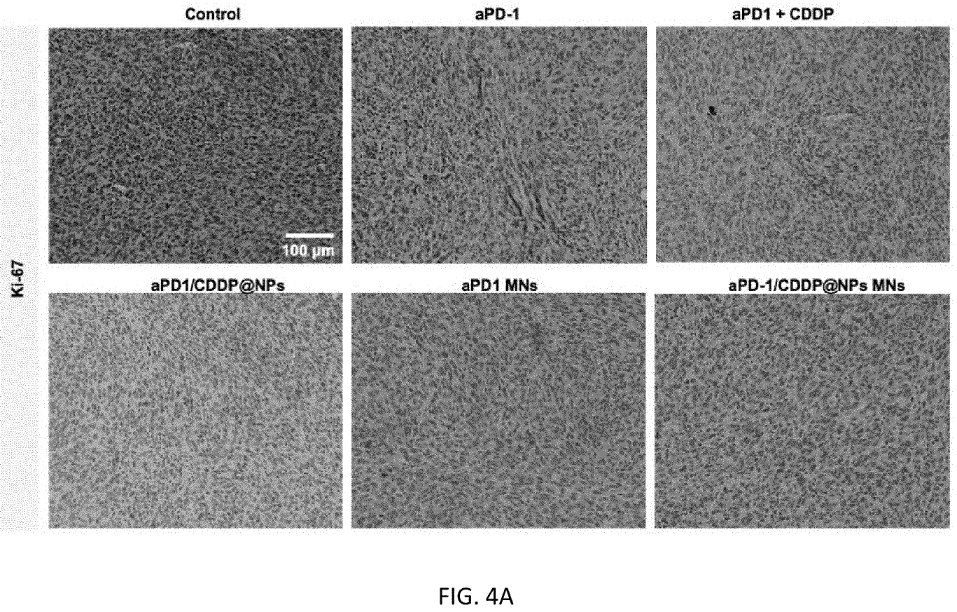

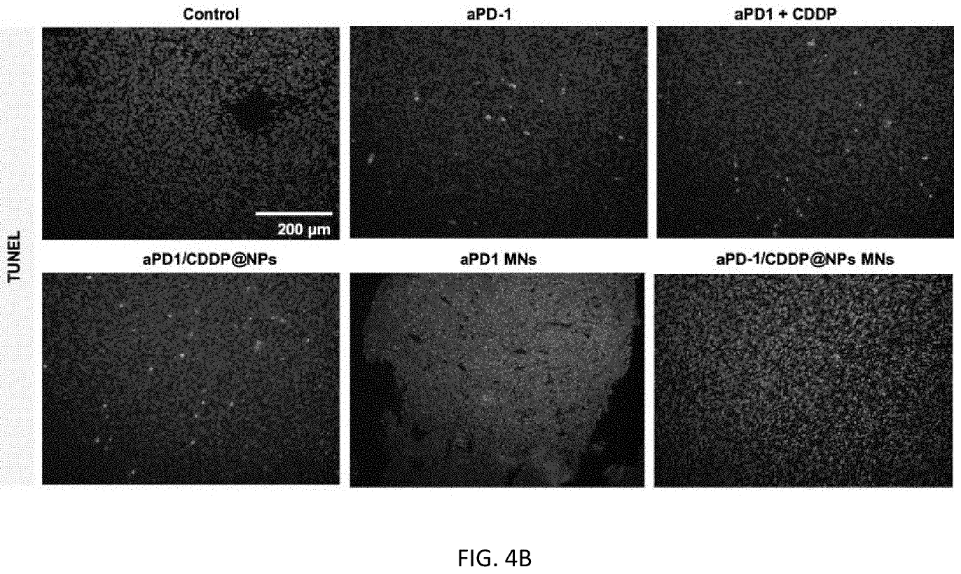

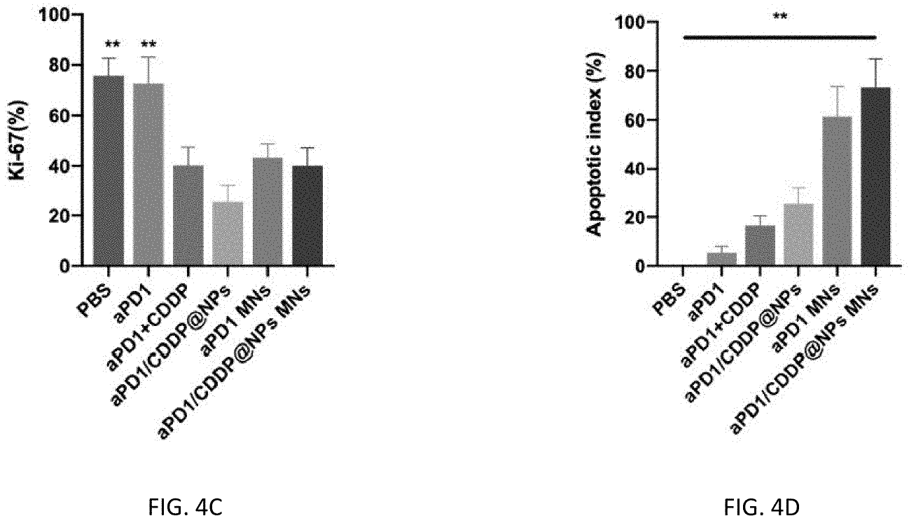

Cell proliferation was further confirmed through IHC in formalin-fixed paraffin-embedded tumor tissue sections. Mice treated with CDDP achieved delayed tumor growth, with 25% of the cells continuing to proliferate, however, mice treated with aPD-1 still exhibited sufficient cell proliferation of approximately 70%. Tumor infiltrating lymphocytes (TILs) like CD4+ cells and CD8+ infiltrated the tumor tissue and started to proliferate after aPD-1 treatment. From FIGS. 4A & 4C, we concluded that CDDP could inhibit cell proliferation, and aPD-1 could enhance T-cell infiltration. aPD-1 delivered through MN patches could amplified the results. TILs attacked the tumor tissues and killed the tumor cells afterwards. Whether these proliferated cells were T-cells or tumor cells were further confirmed through flow cytometry. Additionally, tumor cell apoptosis was assessed through in situ TUNEL assay (FIG. 4B & 4D). The cell apoptotic indexes in the aPD-1 MN and aPD-1-CDDP-NPs MNs groups were 61.4%±12.1% and 73.2%±11.6%, respectively, which were significantly higher than those in the CDDP group (14.4%±5.3%, p<0.01) and aPD-1 group (5.4%±2.7%, p<0.01). These data further substantiated our hypothesis that MN could provoke T-cell activity and then induce cell apoptosis though T-cells. These results demonstrated that the tumor inhibition was mediated through tumor cell apoptosis. In sum, aPD-1-CDDP-NPs MN can not only increase the amount of activated T-cells but also kill tumor cells.

### 6.6 T-cell Responses and Immune Profile

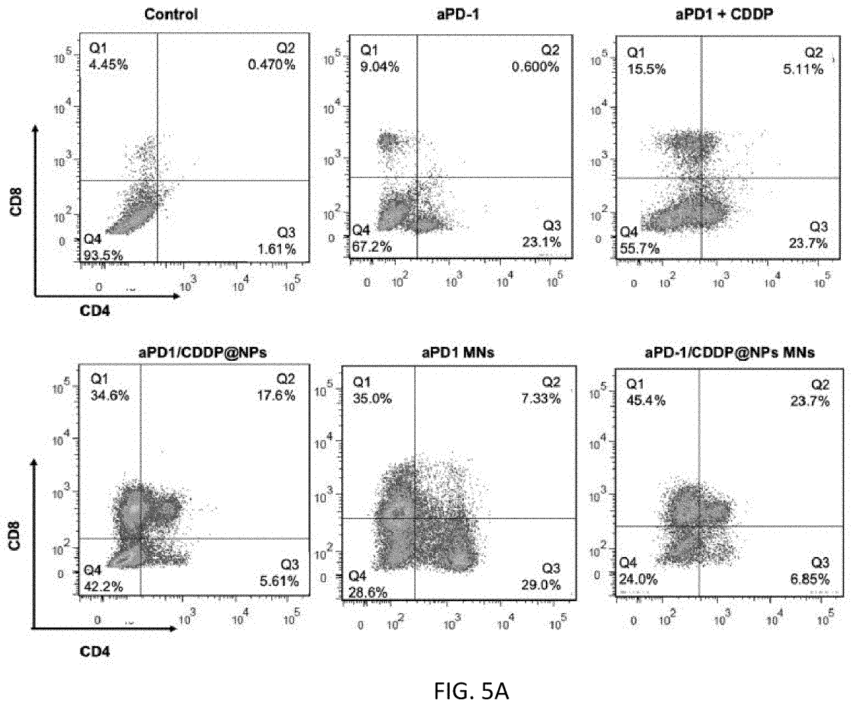

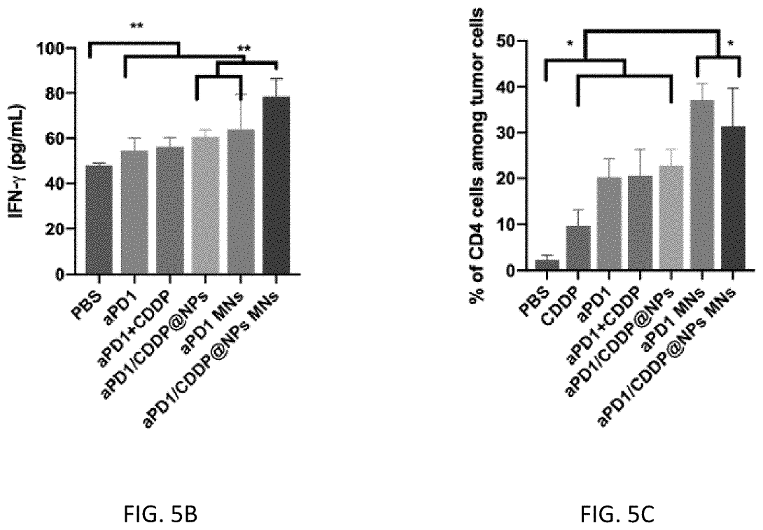

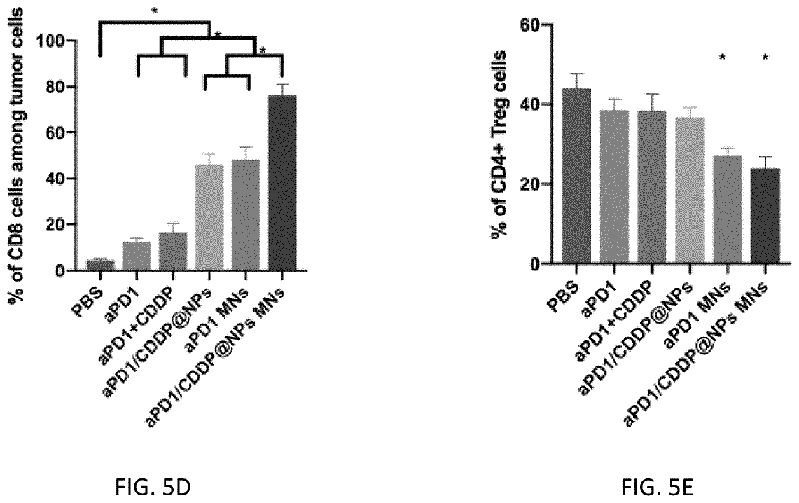

To evaluate T-cell responses and the immune profile, tumor tissues and blood were harvested after sacrifice. aPD-1, being an ICB, could be a positive regulator of TILs. The infiltration of CD4+ T-cells and CD8+ T-cells into tumor cells was assessed through flow cytometry. As illustrated in FIG. 5D, only 4.92% and 4.478% CD8+ T-cells were detected in control and CDDP groups respectively. By contrast, mice treated with aPD-1 or aPD-1+ CDDP groups demonstrated 4-fold T-cells infiltration compared with that in the control group. In addition, significantly increased activated CD8+ T-cells and CD4+ T-cells were detected in both the aPD-1-CDDP-NPs (45.95% for CD8+ and 24.31% for CD 4+) and aPD-1 MN groups (47.98% for CD8+ and 37.50% for CD4+), whereas only 12.13% of CD8+ T-cells and 15.0% CD4+ T-cells were detected in the aPD-1 group, indicating that tumor cells were remarkably infiltrated by T-cells after nano-encapsulation or delivered transdermally. More importantly, MN mediated aPD-1-CDDP-NPs exhibited the highest T-cell infiltration (75.95% of CD8+ T-cells among tumor cells) compared with the other groups. These results were consistent with the IHC-detected Ki-67. The increase of effect T-cells was correlated with the tumor regression efficiency, indicating that activated T-cells were attacking the tumor cells. To reveal the cellular mechanism in vivo, the production of IFN-γ and TNF-α was determined using ELISA kit in mice serum. No significant difference was detected in TNF-α levels; however, IFN-γ demonstrated a positive correlation with CD8+ T-cells (FIG. 5B). Mice treated with PBS and CDDP showed lower IFN-γ levels than other five groups. aPD-1 resulted in more IFN-γ production after nano-encapsulation. Notably, the MN-mediated delivery system could contribute more to IFN-γ expression; among the groups aPD-1-CDDP-NPs MNs group showed the highest IFN-γ expression. Moreover, the infiltration of CD4+Foxp3+ T-cells was analyzed, and the three MN groups showed remarkable decrease of the regulatory T-cells. (FIG. 5E)

Taken together, increased cytotoxic T-cell responses activated by MNs might explain why MN loaded with aPD-1 or aPD-1-CDDP-NPs demonstrated potent anticancer effects in the animal model unresponsive to aPD-1 systemic therapy. The aPD-1-CDDP-NPs delivered by MN patches could robust more T-cells and then infiltrate into the tumor site as well as release chemotherapeutic drugs into the tumor site, leading to synergistic anticancer efficiency.

### 6.7 Systemic Toxicity and Side Effects

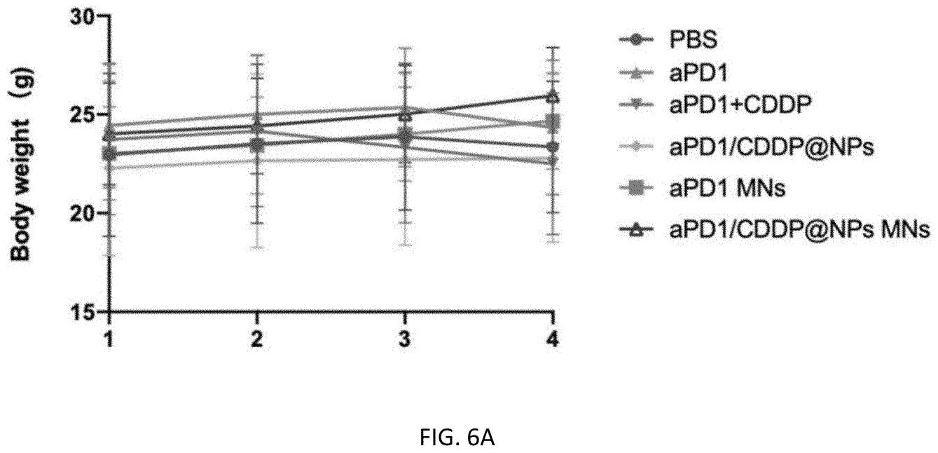

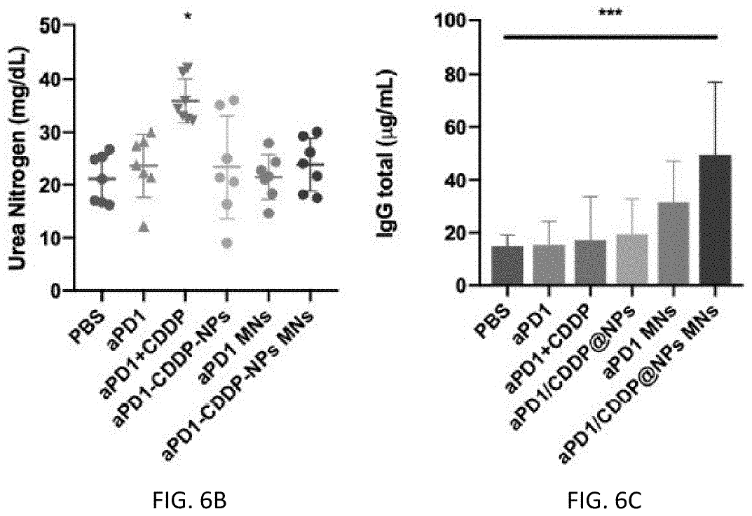

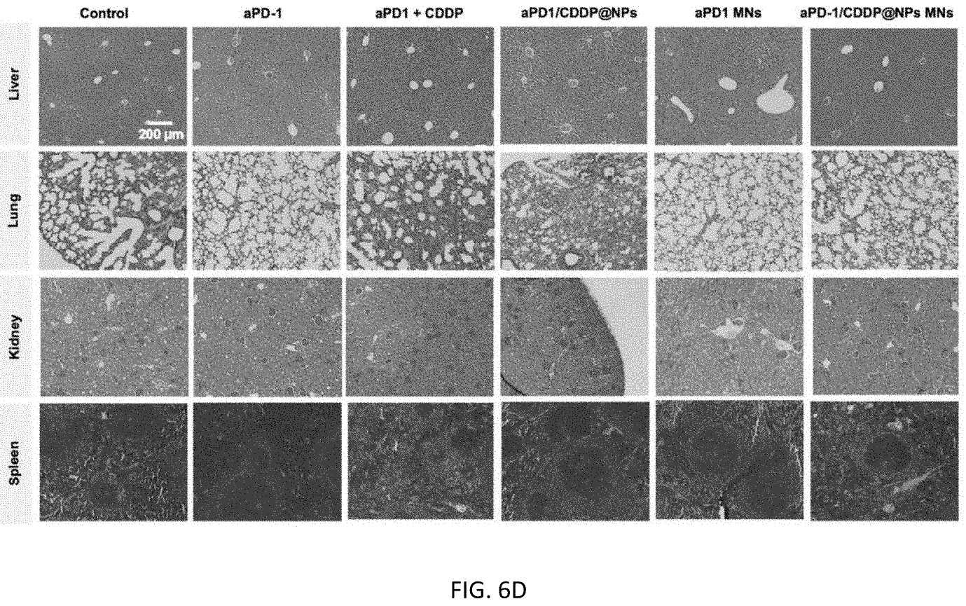

Side effects of chemotherapeutic agents severely limit their clinical application and further lead to non-ideal outcomes. To assess the systemic toxicity and side effects of these treatments, we recorded the body weight, and determined the blood urea nitrogen (BUN) value and total Immunoglobulin G (IgG) value in serum using a urea nitrogen detection kit and total IgG ELISA kit, respectively. Mice treated with CDDP showed severe body weight loss no matter whether adding aPD1. Mice injected with PBS or CDDP exhibited body weight loss after last injection due to cancer cachexia, while mice treated with MNs had no body weight loss during the whole experiment (FIG. 6A). The normal range for BUN value is 12 mg mL⁻¹ to 33 mg mL⁻¹. As illustrated in FIG. 6B, BUN values in mice treated with CDDP or CDDP and aPD-1 were out of the normal range. After nano-encapsulation, the BUN values of some mice in aPD-1-CDDP-NPs group were in normal range, while there was no statistical significance compared to the aPD-1 plus CDDP group. Remarkably, the BUN values in all the MN patches groups were within the normal range, as well as in the control group, indicating MN may be a safe delivery system without causing nephrotoxicity. In addition, CDDP caused severely decreased IgG, while adding aPD-1 to CDDP could recover the downtrend to some extent. Notably, MN groups showed significantly increased IgG values compared with other groups (p<0.001) (FIG. 6C).

Liver, lung, kidney and spleen of each group were collected after sacrifice for hematoxylin and eosin (H&E) staining to analyze whether drugs induced toxicity to these organs (FIG. 6C). Light microscopy analysis revealed that in mice liver sections, some alterations were found in the liver parenchyma in CDDP and aPD1 plus CDDP groups. Microscopic views displayed some areas of necrotic hepatocytes, inflammatory cells infiltration, bile duct proliferation, and hepatocytes swelling. In addition, congestion was slightly more visible in these two groups than in the control group, and the structure of the hepatic lobule was confused. By contrast, the aPD-1-CDDP-NPs group exhibited generally normal structures with a slight disintegration of the hepatic cords, and the structure of hepatic lobule was not entirely clear. In the aPD-1, aPD-1 MNs, and aPD-1-CDDP-NPs MNs groups, the liver parenchyma was quite comparable with that of the control group. For lung sections, compared with the control group, diffuse damage of the pulmonary alveoli and severe inflammatory infiltration were observed in the CDDP, aPD-1 plus CDDP, and aPD-1-CDDP-NPs groups. Other groups displayed normal microscopic lung structures. Regarding nephrotoxicity, the microscopic examination of kidneys from mice in CDDP group and aPD-1 plus CDDP group revealed severe toxic tubular necrosis. This finding was characterized by the glomeruli collapsing and mesangial cells fusing with surrounding tubules, coupled with collapsed Bowman's capsule, disappearance of the Bowman's space and infiltration of scattered lymphocytes. By contrast, there was no evidence of renal damage in mice treated with aPD-1, aPD-1 MNs, and aPD-1-CDDP-NPs MNs. The morphological features of the kidneys were similar to those of the control group. As for spleen in the CDDP group, the white pulp was evidently reduced, and the periarterial lymphatic sheaths disappeared and were accompanied by more neutrophilic granulocyte infiltration, and the boundary of the red and white pulp was unclear. In the aPD-1+ CDDP and aPD-1-CDDP-NPs groups, the white pulp area was reduced, and periarterial lymphatic sheath lymphocytes decreased. In addition, neutrophilic granulocyte infiltration was visible. The microscopic examination of spleens from mice in the aPD-1, aPD-1 MN and NPs MNs groups did not reveal differences with the control group. In sum, CDDP caused severe side effects to all organs, whereas aPD-1 did not induce toxicity to these organs. After nano-encapsulation, the toxicity of CDDP was reduced to some extent, and no apparent toxicity was observed when aPD-1-CDDP-NPs were delivered using MNs. This demonstrated that the transdermal delivery of aPD-1-CDDP-NPs through MN is a safe solution for cancer therapy.

This pioneering study described a novel strategy for the local co-delivery of chemotherapeutic and immunotherapeutic agents by MNs for synergistic immuno-chemotherapy. Lipid-coated nanoparticles were utilized to facilitate the drug release and tumor-targeting. The platform that was integrated with both immune checkpoint blockade and chemotherapy could boost immune response in the tumor microenvironment and further enhance the inhibition efficiency for tumor cells as well as decrease systemic toxicity. Notably, transdermal delivery using MNs increased the response rate in the animal model unresponsive to aPD-1 systemic therapy. Taken together, the synergistic effects of MN-mediated aPD-1-CDDP-NPs render a powerful tool for cancer therapy.

### 7 Materials and Methods

### 7.1 Materials

CDDP (cis-Diammineplatinum(II)dichloride), cyclohexane, Igepal®-520, Triton™ X-100, hexanol, and silver nitrate were purchased from Sigma-Aldrich (St. Louis, Mo., USA). The lipids, DOPA (1,2-dioleoyl-sn-glycerol-3-phospate), DOTAP (1,2-dioleoyl-3-trimethy-lammonium-propane), Liss Rhod PE (1,2-dioleoyl-sn-glycero-3-phosphoethanolamine-N-(lissamine rhodamine B sulfonyl) (ammonium salt)), DSPE-PEG-AA (1,2-distearoyl-sn-glycero-3-phosphoethanolamine-N-[amino(polyethylene glycol)-2000] (ammonium salt)), and cholesterol were obtained from Avanti Polar Lipids (Alabaster, Ala., USA). aPD-1 antibody (GolnVivo™ Purified anti-mouse CD279, Biolegend, San Diego, Calif., USA) was purchased from BioLegend.

### 7.2 Cell Line

FaDu and CAL 27 cell lines, which are human HNSCC cell lines, were obtained from the American Type Culture Collection (ATCC, Manassas, Va., USA). RAW264.7, a type of mouse macrophage cell line, was also obtained from the ATCC. The SCC VII cell line is derived from murine oral squamous cell carcinoma and can be used to build an immunocompetent murine tumor homograft model tumor model on syngeneic hosts ³¹. The SCC VII cell line was kindly gifted by Dr. Susan J Knox from Stanford University, USA. FaDu, CAL 27 and SCC VII cell lines were both cultured in Dulbecco's Modified Eagle's Medium (DMEM, Gibco, Grand Island, N.Y., USA) supplemented with 10% (V:V) fetal bovine serum (FBS, Gibco, USA) and 100 U mL⁻¹ penicillin-streptomycin (Gibco, USA) in a humidified atmosphere with 5% CO2 at 37° C. The RAW 264.7 cell line was maintained in DMEM with 10% FBS without antibiotics and the SCC VII cell line was tagged with the luciferase gen reporter luc2 using the pGL4.51[luc2/CMV/Neo] vector (Promega, Madison, Wis., USA). The gene reporter was transfected using the Lipofectamine 3000 transfection reagent (Thermo Fisher Scientific, Waltham, Mass., USA) following the product protocol. The Luciferase Assay System (Promega, USA) was applied to detection of firefly luciferase activity rapidly in cell lysates. After 14 days of selection with geneticin (Thermo Fishero Scientific, USA) at the concentration of 500 μg mL⁻¹, the luciferase-tagged SCC VII (SCC VII-luc) cell line could be injected into mice and further detected based on bioluminescent signals.

### 7.3 Synthesis of aPD1-CDDP-NPs

Synthesis of the lipid-coated cisplatin nanoparticles (CDDP-NPs) was according to our previous publication.²⁰ Briefly, 0.2 mmol CDDP was dissolved in 1.0 mL deionized (DI) water, and 0.39 mmol silver nitrate was added into the solution. After heating at 60° C. for 3 h, the mixture was stirred overnight protected from light subsequently. After centrifugation at 16000 rpm for 15 min, the supernatant was filtered through a 0.22 μm syringe filter. The concentration of platinum (Pt), monitored through inductively coupled plasma optical emission spectrometry (ICP-OES, Spectro Arcos, Kleve, Germany), was then adjusted to 200 mM; thus the cis-[Pt(NH₃)₂(H₂O)₂]₂(NO₃)₂ precursor was obtained; 800 mM KCl in water and 200 mM cis-[Pt(NH₃)₂(H₂O)₂]₂(NO₃)₂ were added into a microemulsion composed of cyclohexane/Igepal® CO-520 (71%: 29%, V: V) and cyclohexane/Triton™ X-100/hexanol (75%:15%:10%, V:V:V) (3:1) respectively. Subsequently, 20 mM DOPA was added into the precursor solution and stirred for 20 minutes before the two solutions were mixed. Thirty minutes later, ethanol was added into the solution and centrifuged at 12000 g for more than 15 min. After washing with ethanol for at least three times, the particles were re-dispersed into chloroform and lipids, such as DOTAP, DSPE-PEG-AA, and cholesterol, were added into the solution. Subsequently, chloroform was evaporated, and the particles were dispersed into DI water. Ultimately, 100 μg aPD-1 was added into 1.0 mL NPs stirred at 4° C. overnight to obtain the aPD-1-CDDP-NPs. The NPs were collected through centrifugation and followed by re-suspending in DI water.

### 7.4 Characterization of aPD-1-CDDP-NPs

The morphology of aPD-1-CDDP-NPs was observed using a transmission electron microscope (TEM, CM100, Philips Electron Optics, Eindhoven, Netherlands). The particle size was detected through a dynamic light scattering particle size analyzer (Nanotrac Wave II, Microtrac, Montgomeryville, Pa., USA). The CDDP loading capacity was determined based on the Pt content and the Pt content was monitored through ICP-OES. In addition, the loading capacity of aPD-1 was detected using a rat total IgG ELISA kit (Thermo Fisher Scientific, USA). To measure the in vitro aPD-1 release profile, NPs were added to phosphate-buffered saline (PBS) with various pH values of 4.1 and 7.4 at 37° C. Moreover, 10 μL medium was collected for testing, and 10 μL fresh medium was added at a predetermined time point. The amount of aPD-1 was determined using a rat total IgG ELISA kit (Thermo Fisher Scientific, USA).

### 7.5 Antitumor Activity of aPD-1-CDDP-NPs In Vitro

### 7.5.1 Cell Toxicity

FaDu, CAL 27, and SCC VII cell lines were seeded into a 96-well plate at a density of 1×10⁴ cells per well and cultured for 24 h. Cells were then treated with CDDP, aPD-1, L CDDP-NPs, or aPD-1-CDDP-NPs at various concentrations for another 24 h. The cell viability of the treated cells was determined using the Cell Counting Kit-8 (CCK-8, Dojindo, Tokyo, Japan), and the IC50 was calculated using the SPSS software (v.24.0, IBM SPSS, Chicago, Ill., USA).

### 7.5.2 Cellular Uptake

To detect cellular uptake, FaDu and SCC VII cell lines were seeded into a 24-well plate with a density of 5×10⁵ cells per well to detect the cellular uptake. Cells were treated with CDDP, CDDP-NPs or aPD-1-CDDP-NPs at a concentration of 100 μM Pt at 37° C. for 4 h. Subsequently, cells were washed twice with phosphate buffer solution (PBS) and lysed using 69% HNO₃. Water was added into the digested solution to dilute the HNO₃ to 2%, and the content of Pt was determined through ICP-OES.

### 7.6 Cell Apoptosis and Cell Cycle

Cells were placed in 6-cm dishes and treated with CDDP and aPD-1-CDDP-NPs, as the Pt concentration was 100 μM. Cell apoptosis was detected using flow cytometry with the APO-BrdU™ tranferase dUTP nick end laveling (TUNEL) Assay Kit (Thermo Fisher Scientific, USA), and the phase of the cell cycle was determined using propidium iodide (PI, Thermo Fisher Scientific, USA) following the manufacturer's protocol. Cells were fixed with 1% paraformaldehyde (PFA) or ethanol before staining, and they were analyzed through flow cytometry (BD FACSVerse™, BD, Franklin Lake, N.J., USA) immediately after staining. All data were analyzed using FlowJo (7.6.1, BD, USA).

### 7.7 Fabrication of Dissolving MNs

Poly-di-methyl siloxane (PDMS, Sylgard 184, Dow Corning, Mich., USA) was employed to produce the MN mold using the injection molding method. PDMS and its collocational curing agent were blended at a weight ratio of 10:1 and then stirred uniformly. The bubbles in the PDMS mixture solution had to be removed through centrifugation at 7000 rmp for 10 minutes. SU-8 masters were placed in the middle bottom of the PDMS solution without bubbles, and the tip of the needle was upward. The SU-8 masters were placed in the PDMS solution were dried at approximately 70° C. for 24 h to form PDMS mold. The PDMS mold was then detached from the SU-8 master molds and was ready to be used as molds for producing MNs. Thereafter, 1.5 g polyvinyl pyrrolidone (PVP, MW˜360 K, Sigma-Aldrich, USA) was dissolved in 10 mL DI water. aPD-1 or aPD-1-CDDP NPs powder of a specified weight was added in the 15% (w/w) PVP solution, and the 0.1 mL mixed PVP solution was placed into the MNs mold and then centrifuged at 4000 rpm for 5 mins to ensure that the mixed solution filled the cavities. The MNs patch was dried at 40° C. for 24 h in an oven with a constant temperature air circulation system. The dried MNs patch was then ready to be peeled off from the mold for the further use.

### 7.8 Expression of PD-L1 In Vitro and In Vivo

The PD-L1 expression of the SCC VII cell line in vitro was detected through western blot and quantitative polymerase chain reaction (qPCR). Lipopolysaccharide (LPS, Escherichia coli LPS, Sigma, USA) stimulated RAW 264.7 cells were selected as a positive control. SCC VII and RAW 264.7 cells were treated with 10 μmL⁻¹ LPS for 4 h. Subsequently, cells were lysed using RIPA Lysis Buffer with Halt Protease and phosphate inhibitor (Thermo Fisher Scientific, USA). The protein amount was determined using the Pierce™ BCA Protein Assay Kit ((Thermo Fisher Scientific, USA). A total of 30 μg protein was loaded onto SDS-PAGE and transferred to the PVDF membrane. After blocking with 5% non-fat milk in Tris-Buffered Saline (TBS) with 0.1% Tween 20, the membrane was incubated with primary antibodies (anti-PD-L1 and anti-β-actin, Abcam, USA) overnight at 4° C. The membrane was then washed three times with TBS/Tween 20 followed by incubated in HRP-conjugated secondary antibody (Cell Signaling Technology, Danvers, Mass., USA) at room temperature for 2 h. Finally, the membrane was observed using the ChemiDoc XRS System (BioRad, Hercules, Calif., USA) following incubation with the Pierce™ ECL Western Blotting Substrate (Thermo Fisher Scientific, USA). Total RNA in cell lysate was isolated using the RNeasy Mini kit (QIAGEN, Hilden, Germany). cDNA was reverse transcribed from RNA following the manufacture's protocol using the SuperScript™ III Reverse Transcriptase (Thermo Fisher Scientific, USA). Then the cDNA was applied to quantitatively synthesize PCR products by the TaqMan® Fast Advanced Master Mix (Thermo Fisher Scientific, USA). PCR amplification was performed using a StepOnePlus™MReal-Time PCR System (Applied Biosystems, Foster City, Calif., USA). The gene expression of PD-L1 was detected and β-actin was applied to normalize the copy numbers for the target gene.

For the in vivo detection of PD-L1 expression, a part of the tumor tissue was harvested after sacrifice and was fixed in 4% PFA. The tissues were then embedded in paraffin and sliced to a thickness of 5 um. IHC was performed after deparaffinization and rehydration. After antigen retrieval, the sections were incubated with primary antibody, rabbit anti-PD-L1 (Abcam, USA) at 4° C. overnight. Then Rabbit Specific HRP/DAB Detection IHC Kit (Abcam, USA) was applied to detect the expression of PD-L1. Hematoxylin was used for counterstaining. Ultimately, sliced sections were visualized using optical microscopy and photography (ECLIPSE LV100POL, Nikon, Tokyo, Japan), and images were analyzed using the Image J software (NIH, Bethesda, Md., USA).

### 7.9 Antitumor Activity of MNs In Vivo

### 7.9.1 Mice and In Vivo Tumor Models

Male and female C3H/HeJ mice were purchased from The Jackson Laboratory (Bar Harbor, Me., USA) and bred in the Laboratory Animal Unit in the University of Hong Kong. All animal procedures were performed in accordance with a protocol approved by the Committee on the Used of Live Animals in Teaching and Research (CULATR, HKU). An immunocompetent murine tumor homograft model was established by injecting 1×10⁶ SCC VII-luc cells suspended in 100 μL Hanks' balanced salt solution (HBSS, Gibco, USA) on the right flank of the mouse. Sixty-four mice were randomly divided into eight groups, including: (i) control group, which was treated with PBS; (ii) CDDP injected intraperitoneally (indicated as CDDP); (iii) intraperitoneal injection with aPD-1 (indicated as aPD-1); (iv) one side injection with CDDP and the other side injected with aPD-1 (indicated as aPD-1+CDDP); (v) aPD-1-CDDP-NPs injection (indicated as NPs); (vi) LCC NPs delivered by MNs (indicated as LCC NPs MN); (vii) aPD-1-loaded MNs (indicated as aPD-1 MNs); (viii) aPD-1-CDDP-NPs loaded MNs (indicated as NPs MNs). The treatments were administered once the tumor volume reached 10 mm³ and each treatment was administered every 3 days and lasted for 3 cycles. Bioluminescence images were collected through the In Vivo Imaging System—Spectrum (Perkin Elmer, Waltham, Mass., USA). The tumor size was monitored by both the bioluminescence signals and the Vernier calipers every 3 days. Tumor volume was calculated as 0.5× long diameter×short diameter². Tumor volume and tumor wet weight were also measured after sacrifice. Tumor proliferation was determined in sliced tumor tissues using anti-Ki67 (Abcam, USA) following the IHC protocol.

### 7.10 Systemic Immune Effect

### 7.10.1 Blood

Blood was collected from mice and maintained in anticoagulative tubes (VACUETTE® Blood Collection Tubes, K3E K3EDTA, Greiner Bio-One International, Australia). Blood was centrifuged at 1500 g for 10 minutes at 4° C., then plasma was transferred into a new Eppendorf. After centrifugation at 2000 g for 15 mins at 4° C., the supernatant was collected to obtain the serum of mice. IFN-γ and TNF-α in the serum were detected using Mouse IFN-γ ELISA kit (Thermo Fisher Scientific, USA) and Mouse TNF-α ELISA kit (Thermo Fisher Scientific, USA), respectively.

### 7.10.2 Tumor Tissues

A piece of tumor was collected from each mouse for flow cytometry. After execution, the tissues were rinsed with PBS and maintained in PBS at 4° C. The tumor tissues were sliced into very small pieces and sieved through 100 μm cell strainer (BD Falcon, USA). Subsequently, the sliced tissue was pressed using the plunger end of a syringe. The supernatant was decanted and the pellet resuspended the pellet in Flow Cytometry Staining Buffer (PBS plus 10% FBS). The solution was separated into two Eppendorf tubes with a volume of 300 μL, then 1 μL primary antibody was added into one of the two tubes individually. The three primary antibodies used were rabbit anti-CD4 (Abcam, Cambridge, Mass., USA), rat anti-CD8 (Abcam, USA), and mouse anti-foxp3 (Abcam,USA). Secondary antibodies, namely Alexa Fluor®488 goat anti-rabbit IgG (H&L) (Abcam, USA), goat anti-mouse IgG (H&L)-PE, and Alexa Fluor®647 goat anti-rat IgG (H&L) (Abcam, USA), were diluted into flow cytometry staining buffer at a 1:400 ratio, and incubated at room temperature for 15 mins protected from light after added into samples.

### 7.11 Toxicity and Side Effects Study

The livers, kidneys, spleens and lungs were collected after sacrifice and fixed in 4% PFA. After embedding into paraffin, the tissues were sliced into 5-μm sections for H&E staining. Images of the tissues were collected using a polarized light microscope (ECLIPSE LV100POL, Japan). The BUN and total IgG level in the serum were detected using a urea nitrogen detection kit (Thermo Fisher Scientific, USA) and a mouse IgG total ELISA kit (ThermoFisher Scientific, USA) respectively.

### 7.12 Statistical Analysis

All statistical analyses were performed using IBM SPSS Statistics 25.0, including one-way ANOVA, multiple t tests, or the Mann-Whitney U test. The quantitative data are expressed as mean±standard derivation (SD). A p value of <0.05 was considered statistically significant.

### REFERENCES

1. Naiing, A., Immunotherapy. Springer International Publishing AG: 2017.
2. Topalian, S. L.; Drake, C. G.; Pardoll, D. M., Immune Checkpoint Blockade: A Common Denominator Approach to Cancer Therapy. Cancer cell 2015, 27 (4), 450-61.
3. Ni, L.; Dong, C., New Checkpoints in Cancer Immunotherapy. Immunological reviews 2017, 276 (1), 52-65.
4. Ribas, A.; Hamid, O.; Daud, A.; Hodi, F. S.; Wolchok, J. D.; Kefford, R.; Joshua, A. M.; Patnaik, A.; Hwu, W. J.; Weber, J. S.; Gangadhar, T. C.; Hersey, P.; Dronca, R.; Joseph, R. W.; Zarour, H.; Chmielowski, B.; Lawrence, D. P.; Algazi, A.; Rizvi, N. A.; Hoffner, B.; Mateus, C.; Gergich, K.; Lindia, J. A.; Giannotti, M.; Li, X. N.; Ebbinghaus, S.; Kang, S. P.; Robert, C., Association of Pembrolizumab With Tumor Response and Survival Among Patients With Advanced Melanoma. Jama 2016, 315 (15), 1600-9.
5. Dogan, V.; Rieckmann, T.; Munscher, A.; Busch, C. J., Current Studies of Immunotherapy in Head and Neck Cancer. Clinical Otolaryngology 2017.
6. Aspeslagh, S.; Postel-Vinay, S.; Rusakiewicz, S.; Soria, J. C.; Zitvogel, L.; Marabelle, A., Rationale for Anti-0X40 Cancer Immunotherapy. European Journal of Cancer 2016, 52, 50-66.
7. Sharma, P.; Hu-Lieskovan, S.; Wargo, J. A.; Ribas, A., Primary, Adaptive, and Acquired Resistance to Cancer Immunotherapy. Cell 2017, 168 (4), 707-723.
8. Li, Y.; Yang, T.; Yu, Y.; Shi, N.; Yang, L.; Glass, Z.; Bolinger, J.; Finkel, I. J.; Li, W.; Xu, Q., Combinatorial library of chalcogen-containing lipidoids for intracellular delivery of genome-editing proteins. Biomaterials 2018, 178, 652-662.
9. Huang, A. C.; Postow, M. A.; Orlowski, R. J.; Mick, R.; Bengsch, B.; Manne, S.; Xu, W.; Harmon, S.; Giles, J. R.; Wenz, B.; Adamow, M.; Kuk, D.; Panageas, K. S.;Carrera, C.; Wong, P.; Quagliarello, F.; Wubbenhorst, B.; D′Andrea, K.; Pauken, K. E.; Herati, R. S.; Staupe, R. P.; Schenkel, J. M.; McGettigan, S.; Kothari, S.; George, S. M.; Vonderheide, R. H.; Amaravadi, R. K.; Karakousis, G. C.; Schuchter, L. M.; Xu, X.; Nathanson, K. L.; Wolchok, J. D.; Gangadhar, T. C.; Wherry, E. J., T-cell Invigoration to Tumour Burden Ratio Associated with Anti-PD-1 Response. Nature 2017, 545 (7652), 60-65.
10. Wang, W.; Kryczek, I.; Dostal, L.; Lin, H.; Tan, L.; Zhao, L.; Lu, F.; Wei, S.; Maj, T.; Peng, D.; He, G.; Vatan, L.; Szeliga, W.; Kuick, R.; Kotarski, J.; Tarkowski, R.; Dou, Y.; Rattan, R.; Munkarah, A.; Liu, J. R.; Zou, W., Effector T Cells Abrogate Stroma-Mediated Chemoresistance in Ovarian Cancer. Cell 2016, 165 (5), 1092-1105.
11. Xiao, H.; Qi, R.; Li, T.; Awuah, S. G.; Zheng, Y.; Wei, W.; Kang, X.; Song, H.; Wang, Y.; Yu, Y.; Bird, M. A.; Jing, X.; Yaffe, M. B.; Birrer, M. J.; Ghoroghchian, P. P., Maximizing Synergistic Activity When Combining RNAi and Platinum-Based Anticancer Agents. Journal of the American Chemical Society 2017, 139 (8), 3033-3044.
12. Yu, Y.; Xu, Q.; He, S.; Xiong, H.; Zhang, Q.; Xu, W.; Ricotta, V.; Bai, L.; Zhang, Q.; Yu, Z.; Ding, J.; Xiao, H.; Zhou, D., Recent advances in delivery of photosensitive metal-based drugs. Coordination Chemistry Reviews 2019, 387, 154-179.
13. Park, J. H.; Jang, M.; Tarhan, Y. E.; Katagiri, T.; Sasa, M.; Miyoshi, Y.; Kalari, K. R.; Suman, V. J.; Weinshilboum, R.; Wang, L.; Boughey, J. C.; Goetz, M. P.; Nakamura, Y., Clonal Expansion of Antitumor T cells in Breast Cancer Correlates with Response to Neoadjuvant Chemotherapy. International journal of oncology 2016, 49 (2), 471-8.
14. Fridman, W. H.; Zitvogel, L.; Sautes-Fridman, C.; Kroemer, G., The Immune Contexture in Cancer Prognosis and Treatment. Nature Reviews Clinical Oncology 2017, 14 (12), 717-734.
15. Tao, Z.; Muzumdar, M. D.; Detappe, A.; Huang, X.; Xu, E. S.; Yu, Y.; Mouhieddine,
16. T. H.; Song, H.; Jacks, T.; Ghoroghchian, P. P., Differences in Nanoparticle Uptake in Transplanted and Autochthonous Models of Pancreatic Cancer. Nano Letters 2018, 18 (4), 2195-2208.
17. Topalian, S. L.; Weiner, G. J.; Pardoll, D. M., Cancer Immunotherapy Comes of Age. Journal of Clinical Oncology 2011, 29 (36), 4828-36.
18. Ye, Y.; Wang, J.; Hu, Q.; Hochu, G. M.; Xin, H.; Wang, C.; Gu, Z., Synergistic Transcutaneous Immunotherapy Enhances Antitumor Immune Responses through Delivery of Checkpoint Inhibitors. ACS nano 2016, 10 (9), 8956-8963.
19. Wang, C.; Ye, Y.; Hochu, G. M.; Sadeghifar, H.; Gu, Z , Enhanced Cancer Immunotherapy by Microneedle Patch-Assisted Delivery of Anti-PD1 Antibody. Nano Letters 2016, 16 (4), 2334-2340.
20. Xu, J.; Xu, B.; Tao, J.; Yang, Y.; Hu, Y.; Huang, Y., Microneedle-Assisted, DC-Targeted Codelivery of pTRP-2 and Adjuvant of Paclitaxel for Transcutaneous Immunotherapy. Small (Weinheim an der Bergstrasse, Germany) 2017, 13 (28), 1-13.
21. Lan, X.; She, J.; Lin, D.-a.; Xu, Y.; Li, X.; Yang, W.-f.; Lui, V. W. Y.; Jin, L.; Xie, X.; Su, Y.-x., Microneedle-Mediated Delivery of Lipid-Coated Cisplatin Nanoparticles for Efficient and Safe Cancer Therapy. ACS applied materials & interfaces 2018, 10 (39), 33060-33069.
22. Zhang, C.; Jugold, M.; Woenne, E. C.; Lammers, T.; Morgenstern, B.; Mueller, M. M.; Zentgraf, H.; Bock, M.; Eisenhut, M.; Semmler, W.; Kiessling, F., Specific Targeting of Tumor Angiogenesis by RGD-Conjugated Ultrasmall Superparamagnetic Iron Oxide Particles Using A Clinical 1.5-T Magnetic Resonance Scanner. Cancer research 2007, 67 (4), 1555-62.
23. Radwan, A. A.; Alanazi, F. K., Targeting Cancer Using Cholesterol Conjugates. Saudi Pharmaceutical Journal 2014, 22 (1), 3-16.
24. Gibney, G. T.; Weiner, L. M.; Atkins, M. B., Predictive Biomarkers for Checkpoint Inhibitor-Based Immunotherapy. The Lancet. Oncology 2016, 17 (12), e542-e551.
25. Topalian, S. L.; Taube, J. M.; Anders, R. A.; Pardoll, D. M., Mechanism-Driven Biomarkers to Guide Immune Checkpoint Blockade in Cancer Therapy. Nature Reviews Cancer 2016, 16 (5), 275-87.
26. Herbst, R. S.; Soria, J.-C.; Kowanetz, M.; Fine, G. D.; Hamid, O.; Gordon, M. S.; Sosman, J. A.; McDermott, D. F.; Powderly, J. D.; Gettinger, S. N.; Kohrt, H. E. K.; Horn, L.; Lawrence, D. P.; Rost, S.; Leabman, M.; Xiao, Y.; Mokatrin, A.; Koeppen, H.; Hegde, P. S.; Mellman, I.; Chen, D. S.; Hodi, F. S., Predictive Correlates of Response to The Anti-Pd-L1 Antibody MPDL3280A in Cancer Patients. Nature 2014, 515, 563.
27. Lin, E. P.-Y.; Yang, C.-Y.; Lin, C.-W.; Huang, B.-T.; Lai, W.-Y.; Tseng, Y.-T.; Yang, P.-C., Priming PD-L1 Expression by Chemotherapeutic Agents in Non-Small Cell Lung Cancers. Journal of Clinical Oncology 2017, 35 (15_suppl), e20087-e20087.
28. Pol, J.; Vacchelli, E.; Aranda, F.; Castoldi, F.; Eggermont, A.; Cremer, I.; Sautes-Fridman, C.; Fucikova, J.; Galon, J.; Spisek, R.; Tartour, E.; Zitvogel, L.; Kroemer, G.; Galluzzi, L., Trial Watch: Immunogenic Cell Death Inducers for Anticancer Chemotherapy. Oncoimmunology 2015, 4 (4), e1008866.
29. Hugo, W.; Zaretsky, J. M.; Sun, L.; Song, C.; Moreno, B. H.; Hu-Lieskovan, S.; Berent-Maoz, B.; Pang, J.; Chmielowski, B.; Cherry, G.; Seja, E.; Lomeli, S.; Kong, X.; Kelley, M. C.; Sosman, J. A.; Johnson, D. B.; Ribas, A.; Lo, R. S., Genomic and Transcriptomic Features of Response to Anti-PD-1 Therapy in Metastatic Melanoma. Cell 2016, 165 (1), 35-44.
30. Sullivan, S. P.; Koutsonanos, D. G.; del Pilar Martin, M.; Lee, J. W.; Zarnitsyn, V.; Choi, S.-O.; Murthy, N.; Compans, R. W.; Skountzou, I.; Prausnitz, M. R., Dissolving Polymer Microneedle Patches for Influenza Vaccination. Nature Medicine 2010, 16, 915.
31. Esser, E. S.; Pulit-Penaloza, J. A.; Kalluri, H.; McAllister, D.; Vassilieva, E. V.; Littauer, E. Q.; Lelutiu, N.; Prausnitz, M. R.; Compans, R. W.; Skountzou, I., Microneedle Patch Delivery of Influenza Vaccine During Pregnancy Enhances Maternal Immune Responses Promoting Survival and Long-lasting Passive Immunity to Offspring. Scientific reports 2017, 7 (1), 5705-5705.
32. Duarte, S.; Loubat, A.; Momier, D.; Topi, M.; Faneca, H.; Pedroso de Lima, M. C.; Carle, G. F.; Pierrefite-Carle, V., Isolation of Head and Neck Squamous Carcinoma Cancer Stem-Like Cells in A Syngeneic Mouse Model and Analysis of Hypoxia Effect. Oncology Report 2012, 28 (3), 1057-62.

The foregoing description of the specific embodiments will so fully reveal the general nature of the disclosure that others can, by applying knowledge within the skill of the relevant art(s) (including the contents of the documents cited and incorporated by reference herein), readily modify and/or adapt for various applications such specific embodiments, without undue experimentation, without departing from the general concept of the present disclosure. Such adaptations and modifications are therefore intended to be within the meaning and range of equivalents of the disclosed embodiments, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s).

While various embodiments of the present disclosure have been described above, it should be understood that they have been presented by way of examples, and not limitation. It would be apparent to one skilled in the relevant art(s) that various changes in form and detail could be made therein without departing from the spirit and scope of the disclosure. Thus, the present disclosure should not be limited by any of the above-described exemplary embodiments but should be defined only in accordance with the following claims and their equivalents.

All references cited herein are incorporated herein by reference in their entirety and for all purposes to the same extent as if each individual publication or patent or patent application was specifically and individually indicated to be incorporated by reference in its entirety for all purposes.

1. A transdermal drug delivery system comprising microneedles, said microneedles comprises nanoparticles that comprise anti-PD-1-cisplatin.

2. The system of claim 1 wherein the nanoparticles are pH-responsive.

3. The system of claim 1 wherein the nanoparticles comprise a lipid and polyvinylpyrrolidone (PVP).

4. The system of claim 1 wherein the microneedles are water soluble.

5. A method of treatment for cancer in a subject in need thereof comprising administering a transdermal drug delivery system to the subject, said system comprising microneedles and wherein the microneedles comprise a therapeutically effective dose of anti-PD-1-cisplatin-nanoparticles.

6. The method of claim 5 wherein the treatment has a synergistic effect as compared to administration of either anti-PD-1 or cisplatin (CDDP).

7. The method of claim 5 wherein the cancer is melanoma, head and neck cancer or squamous cell carcinoma.

8. The method of claim 5 wherein the method further comprises administration of an additional therapy.

9. The method of claim 5 wherein prior to the treatment, the subject exhibits primary resistance, developed adaptive resistance and/or unresponsive to chemotherapy.

10. The method of claim 5 wherein the treatment boosted an immune response and/or reduced tumor volume by at least 8-fold as compared with systemic injection of anti-PD-1.

11. The method of claim 5 wherein the treatment inhibits cell proliferation and/or enhance T-cell infiltration.
